(19) United States
(12) Patent Application Publication
(10) Pub. No.: US 2018/0014849 A1
(43) Pub. Date: Jan. 18, 2018

(54) SINGLE-USE INSTRUMENT TIP FOR CUSTOMIZED REUSABLE HANDLES

(71) Applicant: Katalyst Surgical, LLC, Chesterfield, MO (US)

(72) Inventors: Gregg D Scheller, Wildwood, MO (US); Eric J Bass, Webster Groves, MO (US)

(73) Assignee: Katalyst Surgical, LLC, Chesterfield, MO (US)

(21) Appl. No.: 15/633,972

(22) Filed: Jun. 27, 2017

Related U.S. Application Data
(60) Provisional application No. 62/363,297, filed on Jul. 17, 2016.

(51) Int. Cl. A61B 17/34; A61B 17/28; A61B 17/3201; A61B 17/00

(52) U.S. Cl. CPC A61B 17/34; A61B 17/3201; A61B 17/28; A61B 2017/0046; A61B 17/3415

(57) ABSTRACT

A single-use instrument tip may include a transitory element, a blank, a hypodermic tube, and a fixation mechanism. The blank may be disposed in the hypodermic tube and the transitory element. A reusable handle may include a handle base, a threaded rod, an instrument tip housing, and an actuation handle. The actuation handle may be manufactured to have one or more properties derived from data collected from a particular surgeon. The actuation handle may include one or more actuation limbs. The threaded rod may be disposed in the handle base, the actuation handle, and the instrument tip housing. The fixation mechanism may be configured to temporarily fix a portion of the single-use instrument tip in the instrument housing. A compression of at least on actuation limb may be configured to extend the hypodermic tube relative to the blank.

### CROSS-REFERENCE TO RELATED APPLICATIONS

This application claims the benefit of U.S. Provisional Application No. 62/363,297, filed Jul. 17, 2016.

### FIELD OF THE INVENTION

The present disclosure relates to a medical device, and, more particularly, to a surgical instrument.

### BACKGROUND OF THE INVENTION

A variety of surgical procedures are performed through a very small surgical incision in a particular tissue. Reducing the size of a surgical incision during a surgical procedure generally reduces the amount of trauma to the surgical site and generally facilitates faster wound healing. In order to perform surgical procedures through a very small surgical incision, a surgeon may require specialized surgical instruments configured to fit through the very small surgical incision and provide the surgeon with a surgical utility. Sometimes a surgeon may require a surgical utility that may not be easily controlled close to a particular surgical site, e.g., closing forceps jaws inside of an eye. It is generally desirable for a surgeon to be able to control such a surgical utility with a minimal amount of effort. For example, if a surgical utility is controlled by a lever or a switch on an instrument handle, a surgeon may need to adjust an orientation of a surgical instrument in order to actuate the lever or the switch. Additionally, if a surgical utility control mechanism requires a surgeon to apply a significant amount of force to a portion of a surgical instrument, then it may be difficult for the surgeon to manipulate the surgical utility control mechanism without unintentionally moving a portion of the surgical instrument.

Ophthalmic surgical instruments are generally categorized as either reusable or single-use. A single-use instrument is typically sterilized prior to sale and is shipped to a surgery center sterile and ready for use in a surgical procedure. A reusable instrument is typically shipped to a surgery center non-sterile and is sterilized by the surgery center between uses in surgery. Reusable instruments are generally lower in overall cost for a surgery center compared to single-use instruments. Single-use instruments offer a surgery center greater convenience compared to reusable instruments. Accordingly, there is a need for an instrument that offers a surgery center the convenience of a single-use instrument at the overall lower cost of a reusable instrument.

### BRIEF SUMMARY OF THE INVENTION

The present disclosure provides a single-use instrument tip for customized reusable handles. Illustratively, a single-use instrument tip may comprise a transitory element, a blank, a hypodermic tube, and a fixation mechanism. In one or more embodiments, the blank may be disposed in the hypodermic tube and the transitory element. Illustratively, a reusable handle may comprise a handle base, a threaded rod, an instrument tip housing, and an actuation handle. In one or more embodiments, the actuation handle may be manufactured to have one or more properties derived from data collected from a particular surgeon. Illustratively, the actuation handle may comprise one or more actuation limbs. In one or more embodiments, a portion of the handle base may be disposed in the actuation handle. Illustratively, the instrument tip housing may be disposed in the actuation handle. In one or more embodiments, the threaded rod may be disposed in the handle base, the actuation handle, and the instrument tip housing. Illustratively, a rotation of the handle base relative to the actuation handle may be configured to rotate the instrument tip housing relative to the actuation handle. In one or more embodiments, the fixation mechanism may be configured to temporarily fix a portion of the single-use instrument tip in the instrument housing. Illustratively, a compression of at least on actuation limb may be configured to extend the hypodermic tube relative to the blank.

### BRIEF DESCRIPTION OF THE DRAWINGS

The above and further advantages of the present invention may be better understood by referring to the following description in conjunction with the accompanying drawings in which like reference numerals indicate identical or functionally similar elements:

FIGS. 1A, 1B, and 1C are schematic diagrams illustrating a transitory element;

FIG. 2 is a schematic diagram illustrating an exploded view of an instrument tip assembly;

FIGS. 3A, 3B, 3C, and 3D are schematic diagrams illustrating an assembled instrument tip;

FIG. 4 is a schematic diagram illustrating an exploded view of a first instrument handle assembly;

FIGS. 5A and 5B are schematic diagrams illustrating an assembled first instrument handle;

FIG. 6 is a schematic diagram illustrating an exploded view of a first instrument assembly;

FIGS. 7A and 7B are schematic diagrams illustrating an assembled first instrument;

FIG. 8 is a schematic diagram illustrating an exploded view of a second instrument handle assembly;

FIGS. 9A and 9B are schematic diagrams illustrating an assembled second instrument handle;

FIG. 10 is a schematic diagram illustrating an exploded view of a second instrument assembly;

FIGS. 11A and 11B are schematic diagrams illustrating an assembled second instrument;

FIG. 12 is a schematic diagram illustrating an exploded view of a third instrument handle assembly;

FIGS. 13A and 13B are schematic diagrams illustrating an assembled third instrument handle;

FIG. 14 is a schematic diagram illustrating an exploded view of a third instrument assembly;

FIGS. 15A and 15B are schematic diagrams illustrating an assembled third instrument.

### DETAILED DESCRIPTION OF AN ILLUSTRATIVE EMBODIMENT

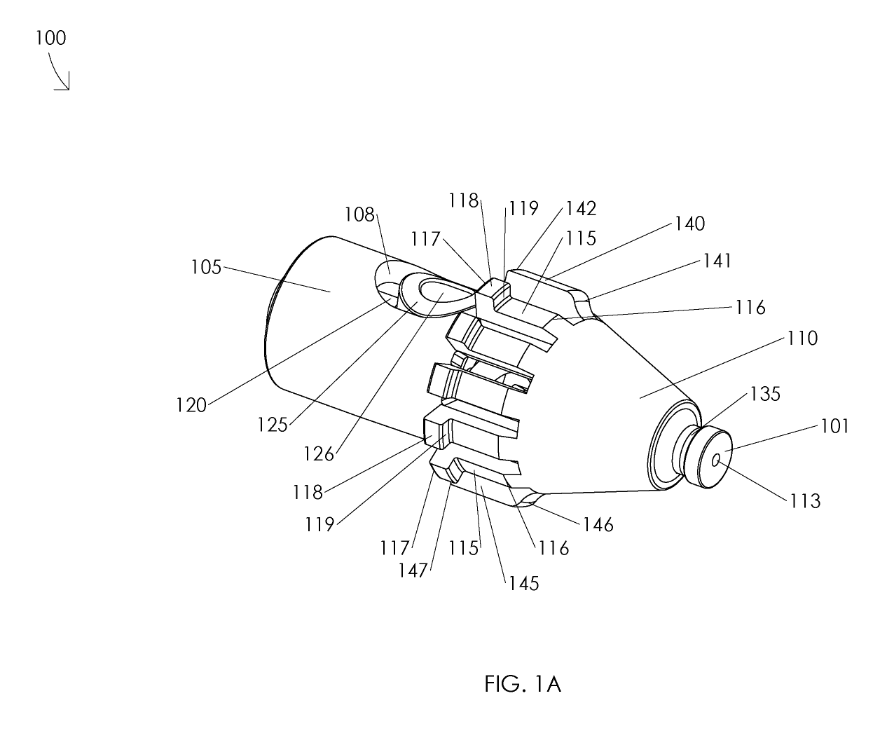

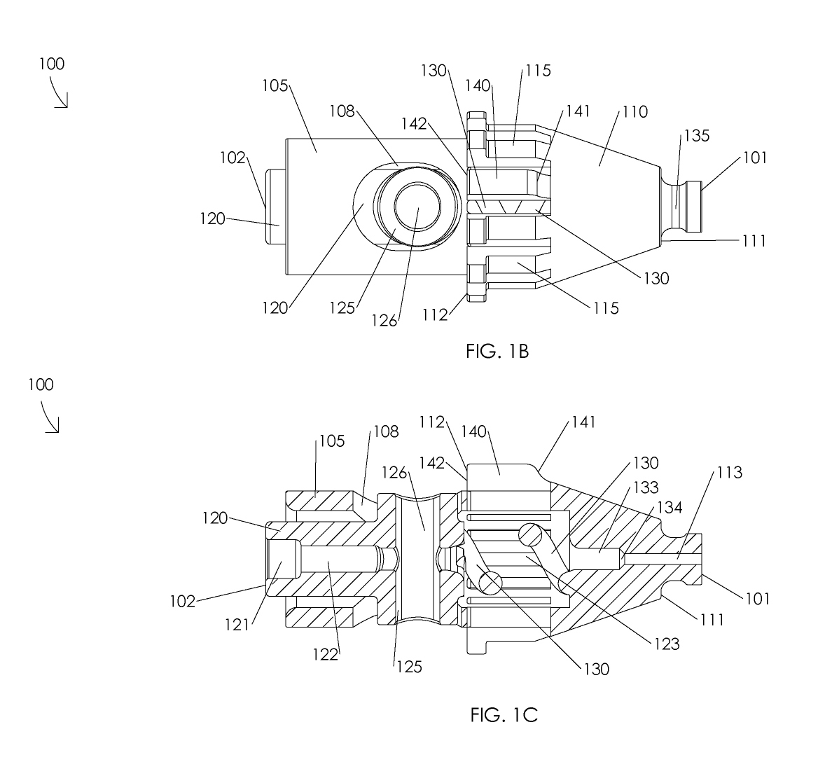

FIGS. 1A, 1B, and 1C are schematic diagrams illustrating a transitory element 100. FIG. 1A illustrates an isometric view of a transitory element 100. FIG. 1B illustrates a superior view of a transitory element 100. FIG. 1C illustrates a cross-sectional view in a sagittal plane of a transitory element 100. In one or more embodiments, a transitory element 100 may comprise a transitory element distal end 101 and a transitory element proximal end 102. Illustratively, transitory element 100 may comprise an outer base 105, a nosecone 110, and a fixation mechanism distal receptacle 120. In one or more embodiments, outer base 105 may comprise an outer base aperture 108. Illustratively, nosecone 110 may comprise a nosecone distal end 111 and a nosecone proximal end 112. In one or more embodiments, nosecone 110 may comprise a hypodermic tube housing 113 and a plurality of radial projections 115. Illustratively, each radial projection 115 of the plurality of radial projections 115 may comprise a radial projection distal end 116 and a radial projection proximal end 117. In one or more embodiments, each radial projection 115 of the plurality of radial projections 115 may be separated from at least one radial projection 115 of the plurality of radial projections 115 by an aperture. Illustratively, nosecone 110 may comprise a plurality of apertures. In one or more embodiments, each radial projection 115 of the plurality of radial projections 115 may comprise a tip cover buttress 118 and a tip cover proximal end interface 119. Illustratively, transitory element 100 may comprise a fixation mechanism support 125 and a fixation mechanism housing 126. In one or more embodiments, transitory element 100 may comprise a fixation mechanism distal receptacle 120. Illustratively, transitory element 100 may comprise an identification ring housing 135. In one or more embodiments, transitory element 100 may comprise a superior projection 140 having a superior projection distal end 141 and a superior projection proximal end 142. Illustratively, transitory element 100 may comprise an inferior projection 145 having an inferior projection distal end 146 and an inferior projection proximal end 147.

In one or more embodiments, transitory element 100 may comprise a fixation mechanism distal housing 121, an inner lumen 122, an inner bore 123, a pressure mechanism 130, a nosecone inner bore 133, and a nosecone inner bore distal taper 134. In one or more embodiments, pressure mechanism 130 may be disposed between inner lumen 122 and hypodermic tube housing 113. Illustratively, inner bore 123 may be disposed in pressure mechanism 130. In one or more embodiments, a portion of pressure mechanism 130 may be disposed in the plurality of radial projections 115. In one or more embodiments, pressure mechanism 130 may be configured to provide a force. Illustratively, pressure mechanism 130 may be configured to provide a constant or uniform force. In one or more embodiments, pressure mechanism 130 may be configured to provide a variable force. Illustratively, pressure mechanism 130 may comprise a spring or a coil. In one or more embodiments, pressure mechanism 130 may comprise a spring having a spring constant in a range of 65.0 to 125.0 pounds per inch, e.g., pressure mechanism 130 may comprise a spring having a spring constant of 94.7 pounds per inch. Illustratively, pressure mechanism 130 may comprise a spring having a spring constant of less than 65.0 pounds per inch or greater than 125.0 pounds per inch. In one or more embodiments, pressure mechanism 130 may comprise a spring having a spring constant in a range of 77.5 to 133.0 pounds per inch, e.g., pressure mechanism 130 may comprise a spring having a spring constant of 107.4 pounds per inch. Illustratively, pressure mechanism 130 may comprise a spring having a spring constant of less than 77.5 pounds per inch or greater than 133.0 pounds per inch. In one or more embodiments, pressure mechanism 130 may comprise a pneumatic system. Illustratively, fixation mechanism support 125 may be disposed in outer base aperture 108, e.g., fixation mechanism support 125 may be configured to actuate within outer base aperture 108. In one or more embodiments, pressure mechanism 130 may be configured to provide a force that resists an actuation of fixation mechanism support 125 within outer base aperture 108. Illustratively, pressure mechanism 130 may be configured to provide a force that facilitates an actuation of fixation mechanism support 125 within outer base aperture 108. In one or more embodiments, nosecone inner bore distal taper 134 may be disposed between nosecone inner bore 133 and hypodermic tube housing 113. Illustratively, nosecone inner bore 133 may be disposed between nosecone inner bore distal taper 134 and inner bore 123.

In one or more embodiments, fixation mechanism distal receptacle 120 may be configured to extend a distance from outer base 105. Illustratively, fixation mechanism distal receptacle 120 may be configured to extend a distance from outer base 105 in a range of 0.02 to 0.06 inches, e.g., fixation mechanism distal receptacle 120 may be configured to extend a distance from outer base 105 of 0.045 inches. In one or more embodiments, fixation mechanism distal receptacle 120 may be configured to extend a distance from outer base 105 of less than 0.02 inches or greater than 0.06 inches. Illustratively, fixation mechanism distal housing 121 may be disposed in fixation mechanism distal receptacle 120. In one or more embodiments, inner lumen 122 may be disposed between fixation mechanism distal housing 121 and pressure mechanism 130. Illustratively, hypodermic tube housing 113 may be disposed between inner bore 123 and nosecone distal end 111.

In one or more embodiments, transitory element 100 may be manufactured from a material configured to deform if transitory element 100 is sterilized in a medical autoclave, e.g., transitory element 100 may be manufactured from a material configured to permanently deform if transitory element 100 is sterilized in a medical autoclave. Illustratively, transitory element 100 may be manufactured from a material having a melting point below a temperature parameter for a steam sterilization cycle, e.g., transitory element 100 may be manufactured from a material having a melting point below a temperature parameter for a gravity-displacement steam sterilization cycle, a dynamic-air-removal steam sterilization cycle, etc. In one or more embodiments, transitory element 100 may be manufactured from a material having a melting point below 140.0 degrees Fahrenheit. Illustratively, transitory element 100 may be manufactured from a material having a melting point in a range of 158.0 to 212.0 degrees Fahrenheit, e.g., transitory element 100 may be manufactured from a material having a melting point of 160.0 degrees Fahrenheit. In one or more embodiments, transitory element 100 may be manufactured from a material having a melting point of less than 158.0 degrees Fahrenheit or greater than 212.0 degrees Fahrenheit. In one or more embodiments, transitory element 100 may be manufactured from a material having a melting point below 250.0 degrees Fahrenheit. Illustratively, transitory element 100 may be manufactured from a material having a melting point below 270.0 degrees Fahrenheit. In one or more embodiments, transitory element 100 may be manufactured from a material having a melting point below 275.0 degrees Fahrenheit.

Illustratively, transitory element 100 may be manufactured from a material configured to temporarily deform if transitory element 100 is sterilized in a medical autoclave, e.g., transitory element 100 may be manufactured from a material configured to absorb water in a medical autoclave. In one or more embodiments, an absorption of water may be configured to deform transitory element 100, e.g., an absorption of water may be configured to cause transitory element 100 to expand. Illustratively, transitory element 100 may be manufactured from a porous material configured to facilitate a deformation of transitory element 100 if transitory element 100 is sterilized in a medical autoclave. In one or more embodiments, transitory element 100 may be manufactured with one or more cavities configured to facilitate a deformation of transitory element 100 if transitory element 100 is sterilized in a medical autoclave. Illustratively, transitory element 100 may be manufactured from any suitable material, e.g., polymers, metals, metal alloys, etc., or from any combination of suitable materials. In one or more embodiments, transitory element 100 may be manufactured by a 3D printing process. For example, transitory element 100 may be manufactured by selective laser sintering, selective heat sintering, selective laser melting, electron-beam melting, direct metal laser sintering, electron beam freeform fabrication, etc. Illustratively, transitory element 100 may be manufactured by injection molding. In one or more embodiments, transitory element 100 may be manufactured by an additive manufacturing process.

In one or more embodiments, transitory element 100 may be manufactured from poly(acrylamide), poly(acrylic acid), poly(adipic anhydride), poly(7-aminoenanthic acid), poly(12-aminolauric acid), poly(11-aminoundecanoic acid), poly(azelaic anhydride), poly[1,3-butadiene(1,4-)-alt-methacrylonitrile], poly[1,3-butadiene(1,4-)-alt-methyl methacrylate], poly(butadiene oxide), poly(caprylaldehyde), poly(1,4-cyclohexylenedimethylene azelate), poly(1,4-cyclohexylenedimethylene dodecanedioate), poly(1,4-cyclohexylenedimethylene glutarate), poly(1,4-cyclohexylenedimethylene p-phenylenediacetate), poly(1,4-cyclohexylenedimethylene pimelate), poly(1,4-cyclohexylenedimethylene sebacate), poly(1,4-cyclohexylenedimethylene suberate), poly(cyclohexylidenethiohexamethylene sulfide), poly(cyclopropylenedimethylene piperazinediurethane), poly(cyclopropylidenedimethylene oxide), poly(decamethylene), poly(decamethylene carbonate), poly[(decamethylenedioxy)-dihexamethylene oxide], poly(decamethylene disulfide), poly(decamethylenedithioethylene disulfide), poly(decamethylenedithiohexamethylene disulfide), poly(decamethylene dithioladipate), poly(decamethylenedithiotetramethylene disulfide), poly(decamethylene pimelate), poly(decamethylene fumaramide), poly(decamethylene glutaramide), poly(decamethylene isophthalate), poly(decamethylene malonate), poly(decamethylene oxydiacetate), poly(decamethyleneoxymethylene oxide), poly(decamethylene succinate), poly(decamethylene sulfide), poly(decamethylene thiodivalerate), poly(decamethylenethiohexamethylene sulfide), poly(divinylbenzal), poly(dodecamethylene), poly(dodecanedioic anhydride), poly(eicosamethylene adipate), poly(eicosamethylene azelate), poly(eicosamethylene glutarate), poly(eicosamethylene isophthalate), poly(eicosamethylene malonate), poly(eicosamethylene oxalate), poly(eicosamethylene oxydiacetate), poly(eicosamethylene phthalate), poly(eicosamethylene pimelate), poly(eicosamethylene sebacate), poly(eicosamethylene suberate), poly(eicosamethylene succinate), poly(eicosamethylene thiodivalerate), poly[ethylene p-(carboxyphenoxy)-butyrate], poly[ethylene p-(carboxyphenoxy)caproate], poly[ethylene p-(carboxyphenoxy)-heptanoate], poly[ethylene p(carboxyphenoxy)-undecanoate], poly[ethylene p-(carboxyphenoxy)-valerate], poly(ethylene 2,2′-dibenzoate), poly[(ethylenedioxy)-diethylene 2,2′-dibenzoate], poly(ethylene 2,2′-dibenzoate), poly[(ethylenedioxy)-diethylene 3,3′-dibenzoate], poly[(ethylenedioxy)-diethylene isophthalate], poly[(ethylenedioxy)-diethylene sebacate], poly[(ethylenedioxy)-diethylene thiodivalerate], poly(ethylene disiloxanylenedipropionamide), poly[(ethylenedithio)-diacetic anhydride], poly[(ethylenedithio)dipropionic anhydride], poly(ethylene dithionisophthalate), poly(ethelene dithiotetramethylene disulfide), poly(ethylene fumaramide), poly(ethylene glutarate), poly(ethylene 2,4-hexadienediamide), poly(ethylene phthalate), poly(ethylene sulfonyldivalerate), poly(ethylene terephthalate), poly(heptamethylene), poly(hexamethylene azelate), poly(hexamethylene carbonate), poly[hexamethylene p-(carboxyphenoxy)-acetate], poly[hexamethylene p-(carboxyphenoxy)-caproate], poly[hexamethylene p(carboxyphenoxy)-undecanoate], poly[hexamethylene p-(carboxyphenoxy)-valerate], poly(hexamethylene isophthalate), poly[hexamethylene (methylene-2,5-tetrahydrofuran)dicarboxamide], poly(hexamethylene octadecanediamide), poly(hexamethylene oxydiacetate), poly(hexamethylene 4,4′-oxydibenzoate), poly(hexamethylene pimelate), poly(hexamethylene succinate), poly(hexamethylene thiodivalerate), poly(hexamethylenethiooentamethylene sulfide), poly(hexamethylenethiotetramethylene sulfide), poly(hexenamer), etc. Illustratively, transitory element 100 may be manufactured from any substituted polymers of poly(acrylamide), poly(acrylic acid), poly(adipic anhydride), poly(7-aminoenanthic acid), poly(12-aminolauric acid), poly(11-aminoundecanoic acid), poly(azelaic anhydride), poly[1,3-butadiene(1,4-)-alt-methacrylonitrile], poly[1,3-butadiene(1,4-)-alt-methyl methacrylate], poly(butadiene oxide), poly(caprylaldehyde), poly(1,4-cyclohexylenedimethylene azelate), poly(1,4-cyclohexylenedimethylene dodecanedioate), poly(1,4-cyclohexylenedimethylene gluestarate), poly(1,4-cyclohexylenedimethylene p-phenylenediacetate), poly(1,4-cyclohexylenedimethylene pimelate), poly(1,4-cyclohexylenedimethylene sebacate), poly(1,4-cyclohexylenedimethylene suberate), poly(cyclohexylidenethiohexamethylene sulfide), poly(cyclopropylenedimethylene piperazinediurethane), poly(cyclopropylidenedimethylene oxide), poly(decamethylene), poly(decamethylene carbonate), poly[(decamethylenedioxy)-dihexamethylene oxide], poly(decamethylene disulfide), poly(decamethylenedithioethylene disulfide), poly(decamethylenedithiohexamethylene disulfide), poly(decamethylene dithioladipate), poly(decamethylenedithiotetramethylene disulfide), poly(decamethylene pimelate), poly(decamethylene fumaramide), poly(decamethylene glutaramide), poly(decamethylene isophthalate), poly(decamethylene malonate), poly(decamethylene oxydiacetate), poly(decamethyleneoxymethylene oxide), poly(decamethylene succinate), poly(decamethylene sulfide), poly(decamethylene thiodivalerate), poly(decamethylenethiohexamethylene sulfide), poly(divinylbenzal), poly(dodecamethylene), poly(dodecanedioic anhydride), poly(eicosamethylene adipate), poly(eicosamethylene azelate), poly(eicosamethylene glutarate), poly(eicosamethylene isophthalate), poly(eicosamethylene malonate), poly(eicosamethylene oxalate), poly(eicosamethylene oxydiacetate), poly(eicosamethylene phthalate), poly(eicosamethylene pimelate), poly(eicosamethylene sebacate), poly(eicosamethylene suberate), poly(eicosamethylene succinate), poly(eicosamethylene thiodivalerate), poly[ethylene p-(carboxyphenoxy)-butyrate], poly[ethylene p-(carboxyphenoxy)caproate], poly[ethylene p-(carboxyphenoxy)-heptanoate], poly[ethylene p(carboxyphenoxy)-undecanoate], poly[ethylene p-(carboxyphenoxy)-valerate], poly(ethylene 2,2′-dibenzoate), poly[(ethylenedioxy)-diethylene 2,2′-dibenzoate], poly(ethylene 2,2′-dibenzoate), poly[(ethylenedioxy)-diethylene 3,3′-dibenzoate], poly[(ethylenedioxy)-diethylene isophthalate], poly[(ethylenedioxy)-diethylene sebacate], poly[(ethylenedioxy)-diethylene thiodivalerate], poly(ethylene disiloxanylenedipropionamide), poly[(ethylenedithio)-diacetic anhydride], poly[(ethylenedithio)dipropionic anhydride], poly(ethylene dithionisophthalate), poly(ethelene dithiotetramethylene disulfide), poly(ethylene fumaramide), poly(ethylene glutarate), poly(ethylene 2,4-hexadienediamide), poly(ethylene phthalate), poly(ethylene sulfonyldivalerate), poly(ethylene terephthalate), poly(heptamethylene), poly(hexamethylene azelate), poly(hexamethylene carbonate), poly[hexamethylene p-(carboxyphenoxy)-acetate], poly[hexamethylene p-(carboxyphenoxy)-caproate], poly[hexamethylene p(carboxyphenoxy)-undecanoate], poly[hexamethylene p-(carboxyphenoxy)-valerate], poly(hexamethylene isophthalate), poly[hexamethylene (methylene-2,5-tetrahydrofuran)dicarboxamide], poly(hexamethylene octadecanediamide), poly(hexamethylene oxydiacetate), poly(hexamethylene 4,4′-oxydibenzoate), poly(hexamethylene pimelate), poly(hexamethylene succinate), poly(hexamethylene thiodivalerate), poly(hexamethylenethiooentamethylene sulfide), poly(hexamethylenethiotetramethylene sulfide), poly(hexenamer), etc.

In one or more embodiments, transitory element 100 may be manufactured by an additive manufacturing process, e.g., transitory element 100 may be manufactured by an additive manufacturing process to eliminate at least one component but retain a functionality of the at least one component. Illustratively, transitory element 100 may be manufactured by an additive manufacturing process to eliminate a spring component but retain the spring component functionality, e.g., pressure mechanism 130 may be configured to retain the spring component functionality. In one or more embodiments, transitory element 100 may be manufactured by an additive manufacturing process to eliminate a lock component but retain the lock component functionality, e.g., fixation mechanism support 125 and fixation mechanism housing 126 may be configured to retain the lock component functionality. Illustratively, transitory element 100 may be manufactured by an additive manufacturing process to eliminate a plurality of components but retain a functionality of component of the plurality of components. In one or more embodiments, transitory element 100 may be manufactured by an additive manufacturing process to eliminate a spring component and a lock component but retain the spring component functionality and the lock component functionality, e.g., pressure mechanism 130 may be configured to retain the spring component functionality and fixation mechanism support 125 and fixation mechanism housing 126 may be configured to retain the lock component functionality. Illustratively, transitory element 100 may be manufactured by an additive manufacturing process to eliminate one or more components wherein eliminating one or more components reduces a cost to manufacture transitory element 100.

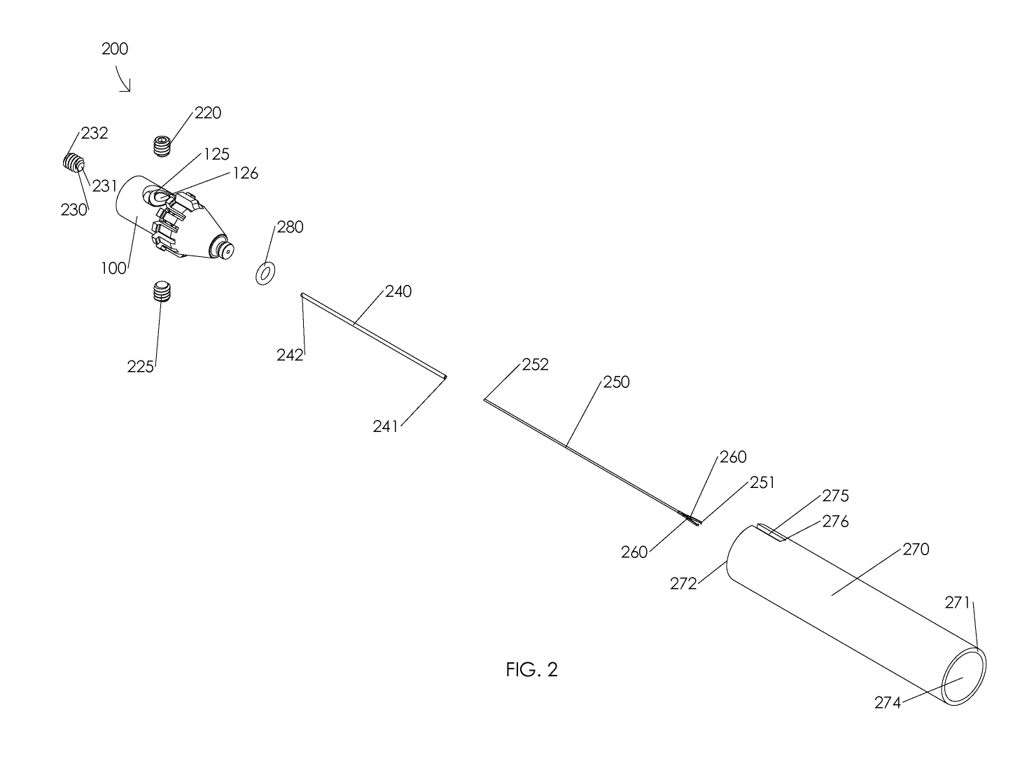

FIG. 2 is a schematic diagram illustrating an exploded view of an instrument tip assembly 200. Illustratively, an instrument tip assembly 200 may comprise a transitory element 100, a superior fixation mechanism 220, an inferior fixation mechanism 225, a fixation mechanism 230, a hypodermic tube 240, a blank 250, a tip cover 270, and an identification ring 280. In one or more embodiments, superior fixation mechanism 220 may comprise a setscrew, a magnet, an adhesive, a weld, etc. Illustratively, inferior fixation mechanism 225 may comprise a setscrew, a magnet, an adhesive, a weld, etc. In one or more embodiments, fixation mechanism 230 may comprise a fixation mechanism distal end 231 and a fixation mechanism proximal end 232. Illustratively, fixation mechanism 230 may comprise a setscrew, a magnet, an adhesive, a weld, etc. In one or more embodiments, hypodermic tube 240 may comprise a hypodermic tube distal end 241 and a hypodermic tube proximal end 242. Illustratively, blank 250 may comprise a blank distal end 251 and a blank proximal end 252. In one or more embodiments, blank 250 may comprise one or more instrument jaws 260, e.g., blank 250 may comprise a pair of instrument jaws 260. Illustratively, blank 250 may comprise a plurality of instrument jaws 260, e.g., blank 250 may comprise two instrument jaws 260, three instrument jaws 260, four instrument jaws 260, five instrument jaws 260, six instrument jaws 260, etc. In one or more embodiments, instrument jaws 260 may comprise forceps jaws, e.g., instrument jaws 260 may be configured to grasp a membrane, an internal limiting membrane, an epiretinal membrane, etc. Illustratively, instrument jaws 260 may comprise scissors blades, e.g., instrument jaws 260 may be configured to cut a tissue. In one or more embodiments, instrument jaws 260 may comprise capsulorhexis forceps jaws, e.g., instrument jaws 260 may be configured to perform a capsulotomy. Illustratively, tip cover 270 may comprise a tip cover distal end 271 and a tip cover proximal end 272. In one or more embodiments, tip cover 270 may comprise a tip cover inner bore 274. Illustratively, tip cover 270 may comprise a first alignment aperture 275 having a first alignment aperture distal end 276 and a second alignment aperture 275 having a second alignment aperture distal end 276.

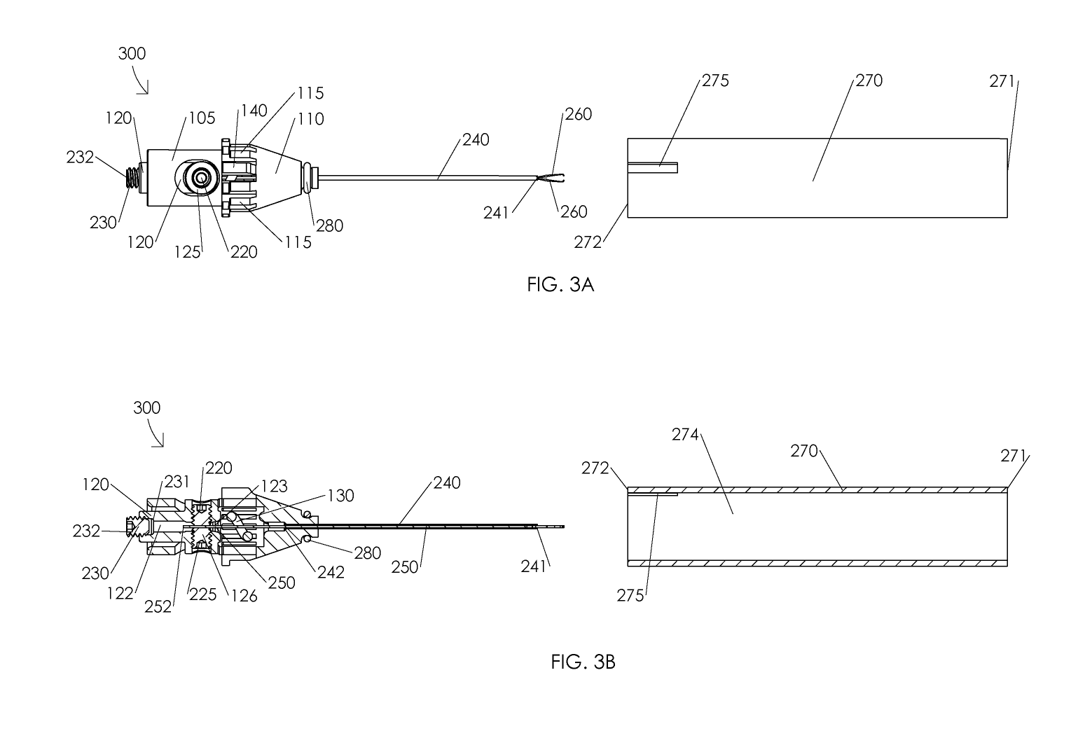

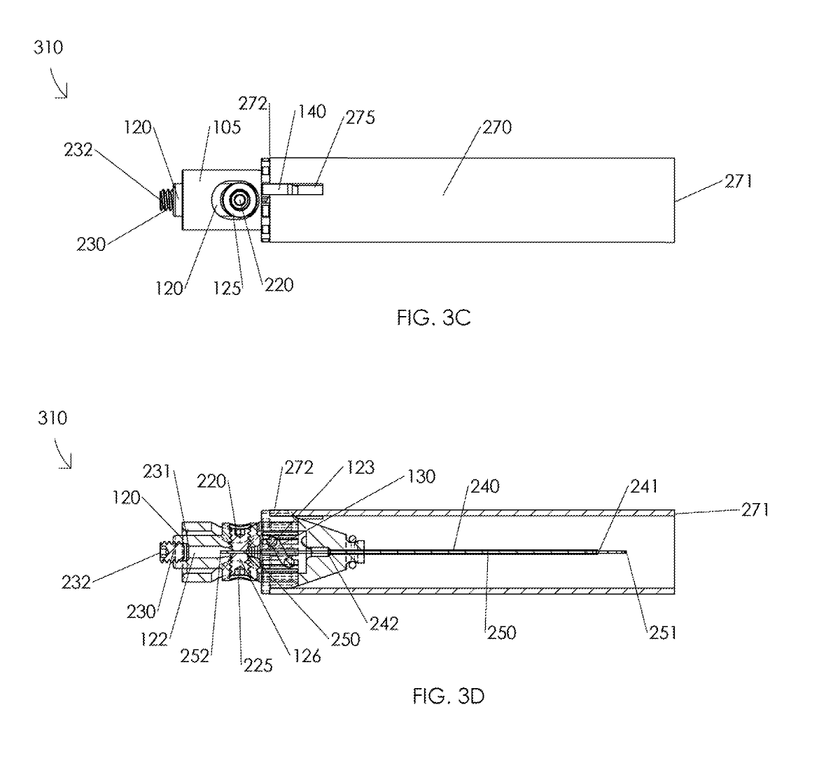

FIGS. 3A, 3B, 3C, and 3D are schematic diagrams illustrating an assembled instrument tip 300. FIG. 3A illustrates a superior view of an assembled instrument tip 300. FIG. 3B illustrates a cross-sectional view in a sagittal plane of an assembled instrument tip 300. FIG. 3C illustrates a superior view of an assembled instrument tip with covered tip 310. FIG. 3D illustrates a cross-sectional view in a sagittal plane of an assembled instrument tip with covered tip 310. Illustratively, a portion of hypodermic tube 240 may be disposed in a portion of nosecone 110, e.g., hypodermic tube proximal end 242 may be disposed in a portion of nosecone 110. In one or more embodiments, a portion of hypodermic tube 240 may be fixed within a portion of nosecone 110, e.g., a portion of hypodermic tube 240 may be fixed within a portion of nosecone 110 by an interference fit, an adhesive, a setscrew, a weld, etc. Illustratively, a portion of hypodermic tube 240 may be disposed in hypodermic tube housing 113, e.g., hypodermic tube proximal end 242 may be disposed in hypodermic tube housing 113 wherein hypodermic tube distal end 241 extends from transitory element distal end 101. In one or more embodiments, a portion of hypodermic tube 240 may be fixed within hypodermic tube housing 113, e.g., a portion of hypodermic tube 240 may be fixed within hypodermic tube housing 113 by an interference fit, an adhesive, a setscrew, a weld, etc.

Illustratively, blank 250 may be disposed in hypodermic tube 240, e.g., blank 250 may be disposed in hypodermic tube 240 wherein blank distal end 251 extends from hypodermic tube distal end 241. In one or more embodiments, blank 250 may be disposed in hypodermic tube 240, nosecone 110, hypodermic tube housing 113, nosecone inner bore distal taper 134, nosecone inner bore 133, inner bore 123, pressure mechanism 130, and fixation mechanism housing 126. Illustratively, superior fixation mechanism 220 may be disposed in fixation mechanism housing 126. In one or more embodiments, inferior fixation mechanism 225 may be disposed in fixation mechanism housing 126. Illustratively, a portion of blank 250 may be disposed between superior fixation mechanism 220 and inferior fixation mechanism 225 within fixation mechanism housing 126. In one or more embodiments, superior fixation mechanism 220 and inferior fixation mechanism 225 may be configured to fix a portion of blank 250 within fixation mechanism housing 126. For example, superior fixation mechanism 220 may comprise a first setscrew and inferior fixation mechanism 225 may comprise a second setscrew. Illustratively, a portion of blank 250 may be fixed in fixation mechanism housing 126 by an interference fit, an adhesive, a setscrew, a weld, etc.

In one or more embodiments, a portion of fixation mechanism 230 may be discs posed in fixation mechanism distal receptacle 120, e.g., fixation mechanism distal end 231 may be disposed in fixation mechanism distal receptacle 120. Illustratively, a first portion of fixation mechanism 230 may be disposed in fixation mechanism distal receptacle 120 wherein a second portion of fixation mechanism 230 extends from transitory element proximal end 102, e.g., fixation mechanism distal end 231 may be disposed in fixation mechanism distal receptacle 120 wherein fixation mechanism proximal end 232 extends from transitory element proximal end 102. In one or more embodiments, a portion of fixation mechanism 230 may be disposed in fixation mechanism distal housing 121. Illustratively, fixation mechanism distal end 231 may be disposed in fixation mechanism distal housing 121 wherein fixation mechanism proximal end 232 extends from transitory element proximal end 102. In one or more embodiments, fixation mechanism distal end 231 may be disposed in fixation mechanism distal housing 121 wherein fixation mechanism proximal end 232 extends a distance from transitory element proximal end 102 in a range of 0.065 to 0.125 inches, e.g., fixation mechanism distal end 231 may be disposed in fixation mechanism distal housing 121 wherein fixation mechanism proximal end 232 extends a distance from transitory element proximal end 102 of 0.094 inches. Illustratively, fixation mechanism distal end 231 may be disposed in fixation mechanism distal housing 121 wherein fixation mechanism proximal end 232 extends a distance from transitory element proximal end 102 of less than 0.065 inches or greater than 0.125 inches. In one or more embodiments, a portion of fixation mechanism 230 may be fixed in fixation mechanism distal housing 121, e.g., fixation mechanism distal end 231 may be fixed in fixation mechanism distal housing 121. Illustratively, a portion of fixation mechanism 230 may be fixed in fixation mechanism distal housing 121 by an interference fit, an adhesive, a magnetic field, a weld, a threading, etc.

In one or more embodiments, identification ring 280 may be disposed over identification ring housing 135, e.g., identification ring 280 may be fixed in identification ring housing 135. Illustratively, identification ring 280 may be configured to indicate one or more properties of assembled instrument tip 300 to a user, e.g., identification ring 280 may be configured to visually indicate one or more properties of assembled instrument tip 300 to a user. In one or more embodiments, identification ring 280 may be configured to indicate a type of cannula that is compatible with assembled instrument tip 300 to a user, e.g., identification ring 280 may be configured to visually indicate a type of cannula that is compatible with assembled instrument tip 300 to a user. Illustratively, identification ring 280 may be configured to indicate a size of cannula that is compatible with assembled instrument tip 300 to a user, e.g., identification ring 280 may be configured to visually indicate a size of cannula that is compatible with assembled instrument tip 300 to a user.

In one or more embodiments, tip cover 270 may be disposed over a portion of transitory element 100 when assembled instrument tip 300 comprises an assembled instrument tip with covered tip 310. Illustratively, tip cover 270 may be disposed over a portion of nosecone 110 when assembled instrument tip 300 comprises an assembled instrument tip with covered tip 310, e.g., tip cover 270 may be disposed over nosecone distal end 111 when assembled instrument tip 300 comprises an assembled instrument tip with covered tip 310. In one or more embodiments, a portion of nosecone 110 may be disposed in tip cover inner bore 274 when assembled instrument tip 300 comprises an assembled instrument tip with covered tip 310, e.g., nosecone distal end 111 may be disposed in tip cover inner bore 274 when assembled instrument tip 300 comprises an assembled instrument tip with covered tip 310. Illustratively, tip cover 270 may be disposed over hypodermic tube 240 when assembled instrument tip 300 comprises an assembled instrument tip with covered tip 310, e.g., tip cover 270 may be disposed over hypodermic tube 240 wherein hypodermic tube distal end 241 is disposed between tip cover distal end 271 and tip cover proximal end 272 and wherein hypodermic tube proximal end 242 is disposed between tip cover distal end 271 and tip cover proximal end 272 when assembled instrument tip 300 comprises an assembled instrument tip with covered tip 310. In one or more embodiments, hypodermic tube 240 may be disposed in tip cover inner bore 274 when assembled instrument tip 300 comprises an assembled instrument tip with covered tip 310, e.g., hypodermic tube 240 may be disposed in tip cover inner bore 274 wherein hypodermic tube distal end 241 is disposed in tip cover inner bore 274 and hypodermic tube proximal end 242 is disposed in tip cover inner bore 274 when assembled instrument tip 300 comprises an assembled instrument tip with covered tip 310. Illustratively, tip cover 270 may be disposed over a portion of blank 250 when assembled instrument tip 300 comprises an assembled instrument tip with covered tip 310, e.g., tip cover 270 may be disposed over blank distal end 251 when assembled instrument tip 300 comprises an assembled instrument tip with covered tip 310. In one or more embodiments, a portion of blank 250 may be disposed in tip cover inner bore 274 when assembled instrument tip 300 comprises an assembled instrument tip with covered tip 310, e.g., blank distal end 251 may be disposed in tip cover inner bore 274 when assembled instrument tip 300 comprises an assembled instrument tip with covered tip 310.

Illustratively, superior projection 140 may be disposed in first alignment aperture 275 when assembled instrument tip 300 comprises an assembled instrument tip with covered tip 310. In one or more embodiments, inferior projection 145 may be disposed in second alignment aperture 275 when assembled instrument tip 300 comprises an assembled instrument tip with covered tip 310. Illustratively, tip cover 270 may be disposed over a portion of radial projection 115 when assembled instrument tip 300 comprises an assembled instrument tip with covered tip 310, e.g., tip cover 270 may be disposed over radial projection distal end 116 when assembled instrument tip 300 comprises an assembled instrument tip with covered tip 310. In one or more embodiments, tip cover 270 may be disposed over a portion of each radial projection 115 of a plurality of radial projections 115 when assembled instrument tip 300 comprises an assembled instrument tip with covered tip 310, e.g., tip cover 270 may be disposed over each radial projection distal end 116 of a plurality of radial projection distal ends 116 when assembled instrument tip 300 comprises an assembled instrument tip with covered tip 310. Illustratively, a portion of radial projection 115 may be disposed in tip cover inner bore 274 when assembled instrument tip 300 comprises an assembled instrument tip with covered tip 310, e.g., radial projection distal end 116 may be disposed in tip cover inner bore 274 when assembled instrument tip 300 comprises an assembled instrument tip with covered tip 310. In one or more embodiments, a portion of each radial projection 115 of a plurality of radial projections 115 may be disposed in tip cover inner bore 274 when assembled instrument tip 300 comprises an assembled instrument tip with covered tip 310, e.g., each radial projection distal end 116 of a plurality of radial projection distal ends 116 may be disposed in tip cover inner bore 274 when assembled instrument tip 300 comprises an assembled instrument tip with covered tip 310.

Illustratively, tip cover proximal end 272 may be adjacent to tip cover proximal end interface 119 when assembled instrument tip 300 comprises an assembled instrument tip with covered tip 310, e.g., tip cover proximal end 272 may abut tip cover proximal end interface 119 when assembled instrument tip 300 comprises an assembled instrument tip with covered tip 310. In one or more embodiments, tip cover proximal end 272 may be adjacent to each tip cover proximal end interface 119 of a plurality of tip cover proximal end interfaces 119 when assembled instrument tip 300 comprises an assembled instrument tip with covered tip 310, e.g., tip cover proximal end 272 may abut each tip cover proximal end interface 119 of a plurality of tip cover proximal end interfaces 119 when assembled instrument tip 300 comprises an assembled instrument tip with covered tip 310. Illustratively, tip cover 270 may be configured to prevent damage to a portion of assembled instrument tip 300 when assembled instrument tip 300 comprises an assembled instrument tip with covered tip 310, e.g., tip cover 270 may be configured to prevent damage to a portion of hypodermic tube 240 when assembled instrument tip 300 comprises an assembled instrument tip with covered tip 310. In one or more embodiments, tip cover 270 may be configured to prevent damage to a portion of blank 250 when assembled instrument tip 300 comprises an assembled instrument tip with covered tip 310, e.g., tip cover 270 may be configured to prevent damage to instrument jaw 260 when assembled instrument tip 300 comprises an assembled instrument tip with covered tip 310.

In one or more embodiments, assembled instrument tip 300 may be a single-use instrument, e.g., assembled instrument tip 300 may be intended for only one use in a surgery. Illustratively, assembled instrument tip 300 may be sterilized after manufacturing but prior to shipment of assembled instrument tip 300 to a user, e.g., assembled instrument tip 300 may be sterilized by ethylene oxide after manufacturing but prior to shipment of assembled instrument tip 300 to a user. In one or more embodiments, one or more properties of assembled instrument tip 300 may be configured to prevent a user from using a sterile assembled instrument tip 300 in a first surgical procedure causing the assembled instrument tip 300 to become non-sterile, sterilizing the assembled instrument tip 300, and using the sterile assembled instrument tip 300 in a second surgical procedure. Illustratively, transitory element 100 may be manufactured from a material configured to deform if transitory element 100 is sterilized in a medical autoclave. In one or more embodiments, transitory element 100 may be manufactured from a material configured to retain ethylene oxide, e.g., transitory element 100 may be manufactured from a material having a degree of crystallinity greater than 60.0 percent. For example, transitory element 100 may be manufactured from a material having a degree of crystallinity greater than 70.0 percent. In one or more embodiments, transitory element 100 may be manufactured from a material having a degree of crystallinity in a range of 60.0 to 80.0 percent, e.g., transitory element 100 may be manufactured from a material having a degree of crystallinity of 75.0 percent. Illustratively, transitory element 100 may be manufactured from a material having a degree of crystallinity of less than 60.0 percent or greater than 80.0 percent. In one or more embodiments, transitory element 100 may be manufactured from a material configured to retain less than 4.0 milligrams of ethylene oxide after a first sterilization by ethylene oxide and configured to retain more than 4.0 milligrams of ethylene oxide after a second sterilization by ethylene oxide. For example, transitory element 100 may be manufactured from polyoxymethylene, polytetrafluoroethylene, isotactic polypropylene, high-density polyethylene, etc. In one or more embodiments, transitory element 100 may be manufactured from a material configured to degrade if transitory element 100 is sterilized by plasma sterilization, e.g., transitory element 100 may be manufactured by a material configured to cross-link in plasma sterilization.

Illustratively, fixation mechanism 230 may be manufactured from a material configured to deform if fixation mechanism 230 is sterilized in a medical autoclave. In one or more embodiments, fixation mechanism 230 may be manufactured from a material having a melting point below 140.0 degrees Fahrenheit. Illustratively, fixation mechanism 230 may be manufactured from a material having a melting point in a range of 158.0 to 212.0 degrees Fahrenheit, e.g., fixation mechanism 230 may be manufactured from a material having a melting point of 160.0 degrees Fahrenheit. In one or more embodiments, fixation mechanism 230 may be manufactured from a material having a melting point of less than 158.0 degrees Fahrenheit or greater than 212.0 degrees Fahrenheit. Illustratively, hypodermic tube 240 may be manufactured from a material configured to deform if hypodermic tube 240 is sterilized in a medical autoclave. In one or more embodiments, hypodermic tube 240 may be manufactured from a material having a melting point below 140.0 degrees Fahrenheit. Illustratively, hypodermic tube 240 may be manufactured from a material having a melting point in a range of 158.0 to 212.0 degrees Fahrenheit, e.g., hypodermic tube 240 may be manufactured from a material having a melting point of 160.0 degrees Fahrenheit. In one or more embodiments, hypodermic tube 240 may be manufactured from a material having a melting point of less than 158.0 degrees Fahrenheit or greater than 212.0 degrees Fahrenheit. Illustratively, blank 250 may be manufactured from a material configured to deform if blank 250 is sterilized in a medical autoclave. In one or more embodiments, blank 250 may be manufactured from a material having a melting point below 140.0 degrees Fahrenheit. Illustratively, blank 250 may be manufactured from a material having a melting point in a range of 158.0 to 212.0 degrees Fahrenheit, e.g., blank 250 may be manufactured from a material having a melting point of 160.0 degrees Fahrenheit. In one or more embodiments, blank 250 may be manufactured from a material having a melting point of less than 158.0 degrees Fahrenheit or greater than 212.0 degrees Fahrenheit.

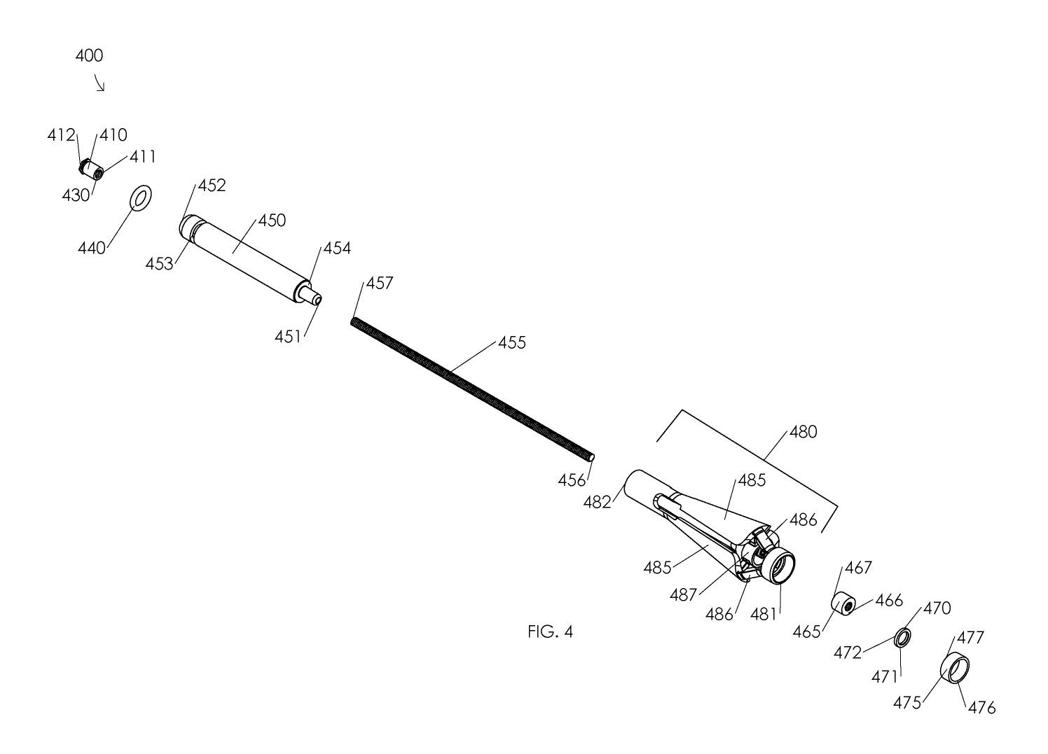

FIG. 4 is a schematic diagram illustrating an exploded view of a first instrument handle assembly 400. In one or more embodiments, a first instrument handle assembly 400 may comprise an end hub 410, a handle identification ring 440, a handle base 450, a threaded rod 455, a first actuation handle 480, an instrument tip housing 465, an instrument tip support ring 470, and a facilitating ring 475. Illustratively, end hub 410 may comprise an end hub distal end 411 and an end hub proximal end 412. In one or more embodiments, end hub 410 may comprise a threaded rod proximal housing 430. Illustratively, handle base 450 may comprise a handle base distal end 451 and a handle base proximal end 452. In one or more embodiments, handle base 450 may comprise a handle identification ring housing 453 and an actuation handle interface 454. Illustratively, threaded rod 455 may comprise a threaded rod distal end 456 and a threaded rod proximal end 457. In one or more embodiments, instrument tip housing 465 may comprise an instrument tip housing distal end 466 and an instrument tip housing proximal end 467. Illustratively, instrument tip support ring 470 may comprise an instrument tip support ring distal end 471 and an instrument tip support ring proximal end 472. In one or more embodiments, facilitating ring 475 may comprise a facilitating ring distal end 476 and a facilitating ring proximal end 477. Illustratively, first actuation handle 480 may comprise a first actuation handle distal end 481 and a first actuation handle proximal end 482. In one or more embodiments, first actuation handle 480 may comprise one or more actuation limbs 485. Illustratively, first actuation handle 480 may comprise a plurality of actuation limbs 485. In one or more embodiments, first actuation handle 480 may comprise one or more extension mechanisms 486. Illustratively, first actuation handle 480 may comprise a plurality of extension mechanisms 486. In one or more embodiments, first actuation handle 480 may comprise at least one extension mechanism 486 for each actuation limb 485. Illustratively, first actuation handle 480 may comprise an inner base 487, e.g., first actuation handle 480 may comprise an inner base 487 disposed between one or more actuation limbs 485. In one or more embodiments, a compression of one or more actuation limbs 485 may be configured to extend first actuation handle distal end 481 relative to first actuation handle proximal end 482. Illustratively, one or more extension mechanisms 486 may be configured to extend first actuation handle distal end 481 relative to first actuation handle proximal end 482. In one or more embodiments, a decompression of one or more actuation limbs 485 may be configured to retract first actuation handle distal end 481 relative to first actuation handle proximal end 482. Illustratively, one or more extension mechanisms 486 may be configured to retract first actuation handle distal end 481 relative to first actuation handle proximal end 482.

In one or more embodiments, first actuation handle 480 may be custom manufactured, e.g., first actuation handle 480 may be manufactured with properties derived from data obtained from a particular surgeon. Illustratively, first actuation handle 480 may be manufactured having an overall outer diameter derived from a particular surgeon's hand dimensions, e.g., a first particular surgeon may have a first particular hand size and a second particular surgeon may have a second particular hand size wherein the second particular hand size is greater than the first particular hand size. In one or more embodiments, a first actuation handle 480 manufactured for the first particular surgeon may have a first overall outer diameter and a first actuation handle 480 manufactured for the second particular surgeon may have a second overall outer diameter wherein the second overall outer diameter is greater than the first overall outer diameter. Illustratively, first actuation handle 480 may be manufactured having an overall length derived from a particular surgeon's hand dimensions, e.g., a first particular surgeon may have a first particular hand size and a second particular surgeon may have a second particular hand size wherein the second particular hand size is greater than the first particular hand size. In one or more embodiments, a first actuation handle 480 manufactured for the first particular surgeon may have a first overall length and a first actuation handle 480 manufactured for the second particular surgeon may have a second overall length wherein the second overall length is greater than the first overall length. Illustratively, first actuation handle 480 may be manufactured to have a particular force applied to one or more actuation limbs 485 to fully extend first actuation handle distal end 481 relative to first actuation handle proximal end 482, e.g., a first surgeon may prefer to apply a first amount of force to one or more actuation limbs 485 to fully extend first actuation handle distal end 481 relative to first actuation handle proximal end 482 and a second surgeon may prefer to apply a second amount of force to one or more actuation limbs 485 to fully extend first actuation handle distal end 481 relative to first actuation handle proximal end 482 wherein the first amount of force is greater than the second amount of force. In one or more embodiments, a first actuation handle 480 manufactured for the first surgeon may have one or more extension mechanisms 486 of a first thickness and a first actuation handle 480 manufactured for the second surgeon may have one or more extension mechanisms 486 of a second thickness wherein the first thickness is greater than the second thickness. Illustratively, first actuation handle 480 may be manufactured from any suitable material, e.g., polymers, metals, metal alloys, etc., or from any combination of suitable materials. In one or more embodiments, first actuation handle 480 may be manufactured by a 3D printing process. For example, first actuation handle 480 may be manufactured by selective laser sintering, selective heat sintering, selective laser melting, electron-beam melting, direct metal laser sintering, electron beam freeform fabrication, etc. Illustratively, first actuation handle 480 may be manufactured by injection molding. In one or more embodiments, first actuation handle 480 may be manufactured by an additive manufacturing process. Illustratively, first actuation handle 480 may be manufacture from a material configured for sterilization in a medical autoclave.

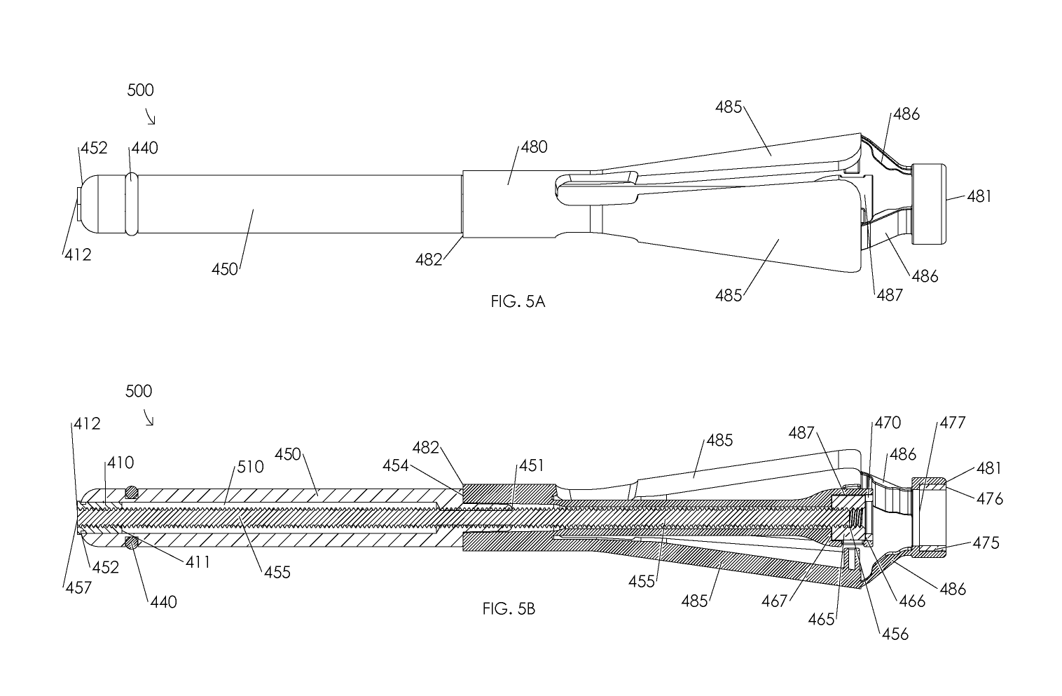

FIGS. 5A and 5B are schematic diagrams illustrating an assembled first instrument handle 500. FIG. 5A illustrates a side view of an assembled first instrument handle 500. FIG. 5B illustrates a cross-sectional in a sagittal plane of an assembled first instrument handle 500. Illustratively, handle base 450 may comprise a handle base inner lumen 510. In one or more embodiments, a portion of threaded rod 455 may be disposed in end hub 410, e.g., threaded rod proximal end 457 may be disposed in threaded rod proximal housing 430. Illustratively, a portion of threaded rod 455 may be disposed in end hub 410 wherein threaded rod proximal end 457 may be adjacent to end hub proximal end 412, e.g., a portion of threaded rod 455 may be disposed in end hub 410 wherein threaded rod proximal end 457 may abut end hub proximal end 412. In one or more embodiments, a portion of threaded rod 455 may be fixed in end hub 410, e.g., a portion of threaded rod 455 may be fixed in end hub 410 by an interference fit, an adhesive, an epoxy, a magnetic field, a weld, a threading, etc. Illustratively, a portion of end hub 410 may be disposed in a portion of handle base 450, e.g., end hub distal end 411 may be disposed in handle base inner lumen 510. In one or more embodiments, a portion of end hub 410 may be disposed in a portion of handle base 450 wherein end hub proximal end 412 extends out from handle base proximal end 452. Illustratively, a portion of end hub 410 may be fixed in a portion of handle base 450, e.g., a portion of end hub 410 may be fixed in a portion of handle base 450 by an interference fit, an adhesive, an epoxy, a magnetic field, a weld, a threading, etc.

In one or more embodiments, a portion of threaded rod 455 may be disposed in handle base 450, e.g., a portion of threaded rod 455 may be disposed in handle base inner lumen 510. Illustratively, a portion of threaded rod 455 may be disposed in handle base 450 wherein threaded rod proximal end 457 extends out from handle base proximal end 452. In one or more embodiments, a portion of threaded rod 455 may be disposed in handle base 450 wherein threaded rod distal end 456 extends out from handle base distal end 451. Illustratively, a portion of threaded rod 455 may be disposed in handle base 450 wherein threaded rod proximal end 457 extends out from handle base proximal end 452 and wherein threaded rod distal end 456 extends out from handle base distal end 451. In one or more embodiments, a portion of threaded rod 455 may be fixed in handle base 450, e.g., a portion of threaded rod 455 may be fixed in handle base 450 by an interference fit, an adhesive, an epoxy, a magnetic field, a weld, a threading, etc.

Illustratively, handle identification ring 440 may be disposed in handle identification ring housing 453. In one or more embodiments, handle identification ring 440 may be fixed in handle identification ring housing 453, e.g., handle identification ring 440 may be fixed in handle identification ring housing 453 by an interference fit, an adhesive, an epoxy, a magnetic field, a weld, a threading, etc. Illustratively, handle identification ring 440 may be configured to indicate one or more properties of assembled first instrument handle 500 to a user, e.g., handle identification ring 440 may be configured to visually indicate one or more properties of assembled first instrument handle 500 to a user. In one or more embodiments, handle identification ring 440 may be configured to indicate a type of cannula that is compatible with assembled first instrument handle 500 to a user, e.g., handle identification ring 440 may be configured to visually indicate a type of cannula that is compatible with assembled first instrument handle 500 to a user. Illustratively, handle identification ring 440 may be configured to indicate a size of cannula that is compatible with assembled first instrument handle 500 to a user, e.g., handle identification ring 440 may be configured to visually indicate a size of cannula that is compatible with assembled first instrument handle 500 to a user. In one or more embodiments, handle identification ring 440 may be configured to correspond to identification ring 280, e.g., a user may compare handle identification ring 440 to identification ring 280 to ensure that a particular assembled instrument tip 300 is configured to connect to a particular assembled first instrument handle 500. Illustratively, a portion of threaded rod 455 may be disposed in a portion of instrument tip housing 465, e.g., threaded rod distal end 456 may be disposed in a portion of instrument tip housing 465. In one or more embodiments, a portion of threaded rod 455 may be fixed in a portion of instrument tip housing 465, e.g., a portion of threaded rod 455 may be fixed in a portion of instrument tip housing 465 by an interference fit, an adhesive, an epoxy, a magnetic field, a weld, a threading, etc.

Illustratively, a portion of handle base 450 may be disposed in a portion of first actuation handle 480, e.g., handle base distal end 451 may be disposed in a portion of first actuation handle 480. In one or more embodiments, a portion of handle base 450 may be disposed in a portion of first actuation handle 480 wherein actuation handle interface 454 may be adjacent to first actuation handle proximal end 482, e.g., a portion of handle base 450 may be disposed in a portion of first actuation handle 480 wherein actuation handle interface 454 may abut first actuation handle proximal end 482. Illustratively, a portion of handle base 450 may be fixed in a portion of first actuation handle 480, e.g., a portion of handle base 450 may be fixed in a portion of first actuation handle 480 by an interference fit, an adhesive, an epoxy, a magnetic field, a weld, a threading, etc. In one or more embodiments, a portion of handle base 450 may be fixed in a portion of first actuation handle 480 by a clearance fit, e.g., a portion of handle base 450 may be fixed in a portion of first actuation handle 480 wherein handle base 450 is configured to rotate relative to first actuation handle 480. Illustratively, a portion of handle base 450 may be fixed in a portion of first actuation handle 480 wherein first actuation handle 480 is configured to rotate relative to handle base 450. In one or more embodiments, a portion of handle base 450 may be fixed in a portion of first actuation handle 480 by an RC1 clearance fit, an RC2 clearance fit, an RC3 clearance fit, an RC4 clearance fit, an RC5 clearance fit, an RC6 clearance fit, an RC7 clearance fit, an RC8 clearance fit, an RC9 clearance fit, etc.

In one or more embodiments, a portion of threaded rod 455 may be disposed in a portion of first actuation handle 480, e.g., threaded rod distal end 456 may be disposed in a portion of first actuation handle 480. Illustratively, a portion of threaded rod 455 may be disposed in a portion of first actuation handle 480 wherein threaded rod distal end 456 may be disposed between first actuation handle distal end 481 and first actuation handle proximal end 482. In one or more embodiments, a portion of threaded rod 455 may be fixed in a portion of first actuation handle 480, e.g., a portion of threaded rod 455 may be fixed in a portion of first actuation handle 480 by an interference fit, an adhesive, an epoxy, a magnetic field, a weld, a threading, etc. Illustratively, a portion of threaded rod 455 may be fixed in a portion of first actuation handle 480 by a clearance fit, e.g., a portion of threaded rod 455 may be fixed in a portion of first actuation handle 480 wherein to threaded rod 455 is configured to rotate relative to first actuation handle 480. In one or more embodiments, a portion of threaded rod 455 may be fixed in a portion of first actuation handle 480 wherein first actuation handle 480 is configured to rotate relative to threaded rod 455. Illustratively, a portion of threaded rod 455 may be fixed in a portion of first actuation handle 480 by an RC1 clearance fit, an RC2 clearance fit, an RC3 clearance fit, an RC4 clearance fit, an RC5 clearance fit, an RC6 clearance fit, an RC7 clearance fit, an RC8 clearance fit, an RC9 clearance fit, etc.

In one or more embodiments, instrument tip housing 465 may be disposed in a portion of first actuation handle 480, e.g., instrument tip housing 465 may be disposed in inner base 487. Illustratively, instrument tip housing 465 may be disposed in a portion of first actuation handle 480 wherein instrument tip housing distal end 466 may be disposed between first actuation handle distal end 481 and first actuation handle proximal end 482. In one or more embodiments, instrument tip housing 465 may be disposed in a portion of first actuation handle 480 wherein instrument tip housing proximal end 467 may be disposed between first actuation handle distal end 481 and first actuation handle proximal end 482. Illustratively, instrument tip housing 465 may be fixed in a portion of first actuation handle 480, e.g., instrument tip housing 465 may be fixed in a portion of first actuation handle 480 by an interference fit, an adhesive, an epoxy, a magnetic field, a weld, a threading, etc. In one or more embodiments, instrument tip housing 465 may be fixed in a portion of first actuation handle 480 by a clearance fit, e.g., instrument tip housing 465 may be fixed in a portion of first actuation handle 480 wherein instrument tip housing 465 is configured to rotate relative to first actuation handle 480. Illustratively, instrument tip housing 465 may be fixed in a portion of first actuation handle 480 wherein first actuation handle 480 is configured to rotate relative to instrument tip housing 465. In one or more embodiments, instrument tip housing 465 may be fixed in a portion of first actuation handle 480 by an RC1 clearance fit, an RC2 clearance fit, an RC3 clearance fit, an RC4 clearance fit, an RC5 clearance fit, an RC6 clearance fit, an RC7 clearance fit, an RC8 clearance fit, an RC9 clearance fit, etc.

In one or more embodiments, instrument tip support ring 470 may be disposed in a portion of first actuation handle 480, e.g., instrument tip support ring 470 may be disposed in inner base 487. Illustratively, instrument tip support ring 470 may be disposed in a portion of first actuation handle 480 wherein instrument tip support ring proximal end 472 may be adjacent to instrument tip housing distal end 456. In one or more embodiments, instrument tip support ring 470 may be disposed in a portion of first actuation handle 480 wherein instrument tip support ring proximal end 472 may abut instrument tip housing distal end 456. Illustratively, instrument tip support ring 470 may be disposed in a portion of first actuation handle 480 wherein instrument tip support ring distal end 471 may be disposed between first actuation handle distal end 481 and first actuation handle proximal end 482. In one or more embodiments, instrument tip support ring 470 may be disposed in a portion of first actuation handle 480 wherein instrument tip support ring proximal end 472 may be disposed between first actuation handle distal end 481 and first actuation handle proximal end 482. Illustratively, instrument tip support ring 470 may be fixed in a portion of first actuation handle 480, e.g., instrument tip support ring 470 may be fixed in a portion of first actuation handle 480 by an interference fit, an adhesive, an epoxy, a magnetic field, a weld, a threading, etc. In one or more embodiments, instrument tip support ring 470 may be fixed in a portion of first actuation handle 480 by a clearance fit, e.g., instrument tip support ring 470 may be fixed in a portion of first actuation handle 480 wherein instrument tip support ring 470 is configured to rotate relative to first actuation handle 480. Illustratively, instrument tip support ring 470 may be fixed in a portion of first actuation handle 480 wherein first actuation handle 480 is configured to rotate relative to instrument tip support ring 470. In one or more embodiments, instrument tip support ring 470 may be fixed in a portion of first actuation handle 480 by an RC1 clearance fit, an RC2 clearance fit, an RC3 clearance fit, an RC4 clearance fit, an RC5 clearance fit, an RC6 clearance fit, an RC7 clearance fit, an RC8 clearance fit, an RC9 clearance fit, etc.

Illustratively, facilitating ring 475 may be disposed in a portion of first actuation handle 480. In one or more embodiments, facilitating ring 475 may be disposed in a portion of first actuation handle 480 wherein facilitating ring distal end 476 may be adjacent to first actuation handle distal end 481, e.g., facilitating ring 475 may be disposed in a portion of first actuation handle 480 wherein facilitating ring distal end 476 may abut first actuation handle distal end 481. Illustratively, facilitating ring 475 may be fixed in a portion of first actuation handle 480, e.g., facilitating ring 475 may be fixed in a portion of first actuation handle 480 by an interference fit, an adhesive, an epoxy, a magnetic field, a weld, a threading, etc. In one or more embodiments, facilitating ring 475 may be fixed in a portion of first actuation handle 480 by a clearance fit, e.g., facilitating ring 475 may be fixed in a portion of first actuation handle 480 wherein facilitating ring 475 is configured to rotate relative to first actuation handle 480. Illustratively, facilitating ring 475 may be fixed in a portion of first actuation handle 480 wherein first actuation handle 480 is configured to rotate relative to facilitating ring 475. In one or more embodiments, facilitating ring 475 may be fixed in a portion of first actuation handle 480 by an RC1 clearance fit, an RC2 clearance fit, an RC3 clearance fit, an RC4 clearance fit, an RC5 clearance fit, an RC6 clearance fit, an RC7 clearance fit, an RC8 clearance fit, an RC9 clearance fit, etc.

Illustratively, a rotation of handle base 450 relative to first actuation handle 480 may be configured to rotate end hub 410 relative to first actuation handle 480. In one or more embodiments, a rotation of end hub 410 relative to first actuation handle 480 may be configured to rotate threaded rod 455 relative to first actuation handle 480. Illustratively, a rotation of handle base 450 relative to first actuation handle 480 may be configured to rotate threaded rod 455 relative to first actuation handle 480. In one or more embodiments, a rotation of threaded rod 455 relative to first actuation handle 480 may be configured to rotate instrument tip housing 465 relative to first actuation handle 480. Illustratively, a rotation of instrument tip housing 465 relative to first actuation handle 480 may be configured to rotate instrument tip support ring 470 relative to first actuation handle 480. In one or more embodiments, a rotation of handle base 450 relative to first actuation handle 480 may be configured to rotate threaded rod 455, end hub 410, instrument tip housing 465, and instrument tip support ring 470 relative to first actuation handle 480.

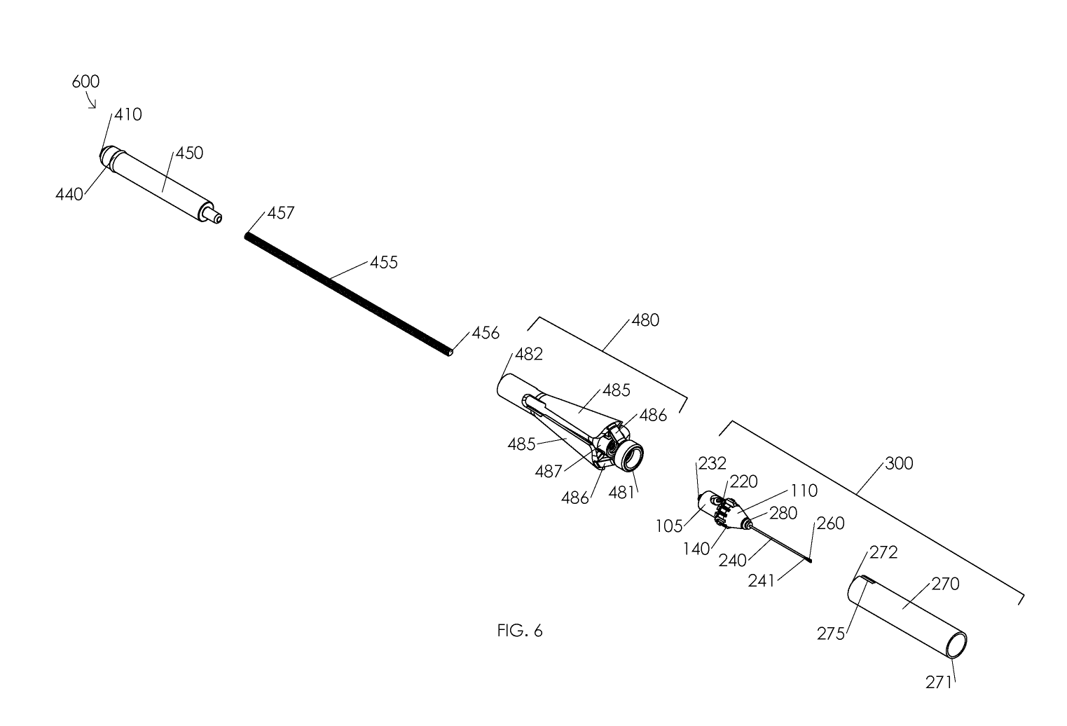

FIG. 6 is a schematic diagram illustrating an exploded view of a first instrument assembly 600. Illustratively, a first instrument assembly 600 may comprise an end hub 410, handle identification ring 440, a handle base 450, a threaded rod 455, a first actuation handle 480, and an assembled instrument tip 300.

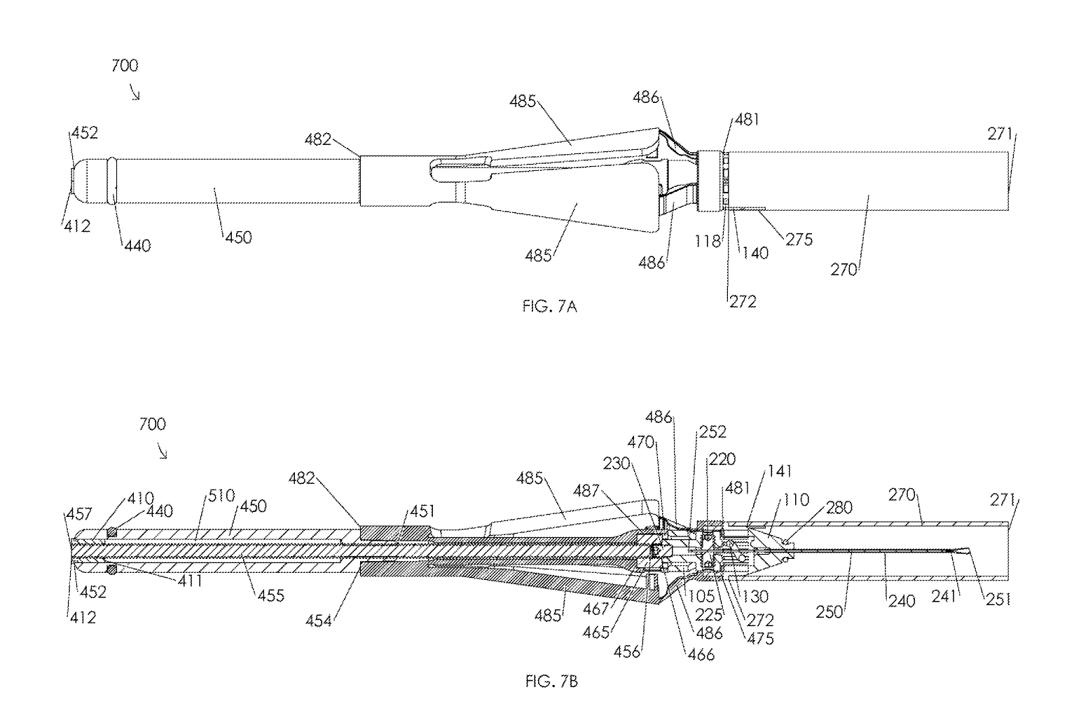

FIGS. 7A and 7B are schematic diagrams illustrating an assembled first instrument 700. FIG. 7A illustrates a side view of an assembled first instrument 700. FIG. 7B illustrates a cross-sectional view in a sagittal plane of an assembled first instrument 700. Illustratively, a portion of fixation mechanism 230 may be disposed in instrument tip housing 465, e.g., fixation mechanism proximal end 232 may be disposed in instrument tip housing 465. In one or more embodiments, a first portion of fixation mechanism 230 may be disposed instrument tip housing 465 wherein a second portion of fixation mechanism 230 may be disposed in fixation mechanism distal receptacle 120, e.g., fixation mechanism proximal end 232 may be disposed in instrument tip housing 465 and fixation mechanism distal end 231 may be disposed in fixation mechanism distal receptacle 120. Illustratively, a first portion of fixation mechanism 230 may be disposed in instrument tip housing 465 wherein a second portion of fixation mechanism 230 may be disposed in fixation mechanism distal housing 121, e.g., fixation mechanism proximal end 232 may be disposed in instrument tip housing 465 and fixation mechanism distal end 321 may be disposed in fixation mechanism distal housing 121. In one or more embodiments, a portion of fixation mechanism 230 may be fixed in instrument tip housing 465, e.g., fixation mechanism proximal end 232 may be fixed in instrument tip housing 465. Illustratively, a portion of fixation mechanism 230 may be fixed in instrument tip housing 465 by an interference fit, an adhesive, an epoxy, a magnetic field, a weld, a threading, etc. In one or more embodiments, a first portion of fixation mechanism 230 may be fixed in instrument tip housing 465 and a second portion of fixation mechanism 230 may be fixed in fixation mechanism distal housing 121, e.g., fixation mechanism proximal end 232 may be fixed in instrument tip housing 465 and fixation mechanism distal end 231 may be fixed in fixation mechanism distal housing 121. In one or more embodiments, instrument tip support ring 470 may be configured to support assembled instrument tip 300, e.g., instrument tip support ring 470 may be configured to prevent assembled instrument tip 300 from vibrating in first actuation handle 480.

Illustratively, fixation mechanism 230 may comprise a setscrew configured to screw into fixation mechanism distal housing 121 and instrument tip housing 465. In one or more embodiments, fixation mechanism 230 may be permanently fixed in fixation mechanism distal housing 121, e.g., fixation mechanism distal end 231 may be fixed in distal housing 121 wherein removing fixation mechanism distal end 231 from distal housing 121 may be configured to damage a portion of transitory element 100. Illustratively, fixation mechanism distal end 231 may be permanently fixed in distal housing 121 wherein removing fixation mechanism distal end 231 from distal housing 121 may be configured to damage fixation mechanism distal receptacle 120. In one or more embodiments, fixation mechanism 230 may be temporarily fixed in instrument tip housing 465, e.g., fixation mechanism proximal end 232 may be fixed in instrument tip housing 465 wherein fixation mechanism proximal end 232 is removable from instrument tip housing 465. Illustratively, a first fixation mechanism proximal end 232 may be temporarily fixed in instrument tip housing 465 wherein the first fixation mechanism proximal end 232 may be removed from instrument tip housing 465 and a second fixation mechanism proximal end 232 may be inserted in instrument tip housing 465. In one or more embodiments, a first fixation mechanism proximal end 232 may be temporarily fixed in instrument tip housing 465 wherein the first fixation mechanism proximal end 232 may be removed from instrument tip housing 465 and a second fixation mechanism proximal end 232 may be temporarily fixed in instrument tip housing 465.

In one or more embodiments, a compression of one or more actuation limbs 485 may be configured to extend first actuation handle distal end 481 relative to first actuation handle proximal end 482. Illustratively, an extension of first actuation handle distal end 481 relative to first actuation handle proximal end 482 may be configured to extend nosecone 110 relative to handle base 105. In one or more embodiments, an extension of nosecone 110 relative to handle base 105 may be configured to extend hypodermic tube 240 relative to blank 250. Illustratively, an extension of hypodermic tube 240 relative to blank 250 may be configured to extend hypodermic tube distal end 241 over a portion of a first instrument jaw 260 and over a portion of a second instrument jaw 260. In one or more embodiments, an extension of hypodermic tube distal end 241 over a portion of a first instrument jaw 260 and over a portion of a second instrument jaw 260 may be configured to reduce a separation distance between the first instrument jaw 260 and the second instrument jaw 260. Illustratively, an extension of nosecone 110 relative to handle base 105 may be configured to expand pressure mechanism 130. In one or more embodiments, pressure mechanism 130 may be configured to provide a force that resists an extension of nosecone 110 relative to handle base 105, e.g., pressure mechanism 130 may be configured to provide a force that facilitates a retraction of nosecone 110 relative to handle base 105.

In one or more embodiments, a decompression of one or more actuation limbs 485 may be configured to retract first actuation handle distal end 481 relative to first actuation handle proximal end 482. Illustratively, a retraction of first actuation handle distal end 481 relative to first actuation handle proximal end 482 may be configured to retract nosecone 110 relative to handle base 105. In one or more embodiments, a retraction of nosecone 110 relative to handle base 105 may be configured to retract hypodermic tube 240 relative to blank 250. Illustratively, a retraction of hypodermic tube 240 relative to blank 250 may be configured to retract hypodermic tube distal end 241 off from a portion of a first instrument jaw 260 and off from a portion of a second instrument jaw 260. In one or more embodiments, a retraction of hypodermic tube distal end 241 off from a portion of a first instrument jaw 260 and off from a portion of a second instrument jaw 260 may be configured to increase a separation distance between the first instrument jaw 260 and the second instrument jaw 260. Illustratively, a retraction of nosecone 110 relative to handle base 105 may be configured to collapse pressure mechanism 130. In one or more embodiments, pressure mechanism 130 may be configured to provide a force that facilitates a retraction of nosecone 110 relative to handle base 105.

Illustratively, a rotation of handle base 450 relative to first actuation handle 480 may be configured to rotate assembled instrument tip 300 relative to first actuation handle 480. In one or more embodiments, a rotation of handle base 450 relative to first actuation handle 480 may be configured to rotate instrument tip housing 465 relative to first actuation handle 480. Illustratively, a rotation of instrument tip housing 465 relative to first actuation handle 480 may be configured to rotate assembled instrument tip 300 relative to first actuation handle 480. In one or more embodiments, first actuation handle 480 may be configured to rotate relative to assembled instrument tip 300. Illustratively, a surgeon may selectively orient first instrument jaw 260 and second instrument jaw 260 in any rotational orientation by rotating handle base 450, e.g., a surgeon may insert first instrument jaw 260 and second instrument jaw 260 through a cannula and into an eye and the surgeon may selectively orient first instrument jaw 260 and second instrument jaw 260 in any rotational orientation within the eye by rotating handle base 450. In one or more embodiments, a surgeon may insert first instrument jaw 260 and second instrument jaw 260 through a cannula and into an eye and the surgeon may selectively orient first instrument jaw 260 and second instrument jaw 260 in any rotational orientation within the eye without rotating first actuation handle 480.

Illustratively, facilitating ring 475 may be configured to facilitate a rotation of assembled instrument tip 300 relative to first actuation handle 480. In one or more embodiments, facilitating ring 475 may be manufactured from a material configured to facilitate a rotation of assembled instrument tip 300 relative to first actuation handle 480. Illustratively, facilitating ring 475 may be manufactured from a material configured to minimize a coefficient of friction between a portion of assembled instrument tip 300 and a portion of facilitating ring 475, e.g., facilitating ring 475 may be manufactured from a material configured to minimize a coefficient of friction between outer base 105 and a portion of facilitating ring 475. In one or more embodiments, facilitating ring 475 may be manufactured from a self-lubricating thermoplastic material. Illustratively, facilitating ring 475 may be manufactured from an oil-impregnated bronze alloy, e.g., facilitating ring 475 may be manufactured from Oilite, SAE 841 bronze, etc. In one or more embodiments, facilitating ring 475 may be manufactured from a material wherein a coefficient of friction between a portion of assembled instrument tip 300 and a portion of facilitating ring 475 is in a range of 0.011 to 0.36, e.g., facilitating ring 475 may be manufactured from a material wherein a coefficient of friction between a portion of assembled instrument tip 300 and a portion of facilitating ring 475 is 0.0311. Illustratively, facilitating ring 475 may be manufactured from a material wherein a coefficient of friction between a portion of assembled instrument tip 300 and a portion of facilitating ring 475 is less than 0.011 or greater than 0.36. In one or more embodiments, facilitating ring 475 may be manufactured from a fluorocarbon material, e.g., facilitating ring 475 may be manufactured from a polytetrafluoroethylene material. Illustratively, facilitating ring 475 may be manufactured from an acetal-based polytetrafluoroethylene material, e.g., facilitating ring 475 may be manufactured from a turcite material. In one or more embodiments, facilitating ring 475 may be manufactured from a material having a density in a range of 0.024 to 0.073 pounds per cubic inch, e.g., facilitating ring 475 may be manufactured from a material having a density of 0.053 pounds per cubic inch. Illustratively, facilitating ring 475 may be manufactured from a material having a density of less than 0.024 pounds per cubic inch or greater than 0.073 pounds per cubic inch. In one or more embodiments, facilitating ring 475 may be manufactured from a material having a hardness in a range of 50 Shore D to 75 Shore D, e.g., facilitating ring 475 may be manufactured from a material having a hardness of 61 Shore D. Illustratively, facilitating ring 475 may be manufactured from a material having a hardness of less than 50 Shore D or greater than 75 Shore D. In one or more embodiments, transitory element 100 may be manufactured from a material having a first hardness and facilitating ring 475 may be manufactured from a material having a second hardness. Illustratively, the first hardness may be greater than the second hardness.

Illustratively, assembled first instrument handle 500 may comprise a reusable instrument and assembled instrument tip 300 may comprise a single-use instrument. In one or more embodiments, a user may install a first assembled instrument tip 300 in assembled first instrument handle 500 by inserting fixation mechanism proximal end 232 into instrument tip housing 465, e.g., a user may install a first assembled instrument tip 300 in assembled first instrument handle 500 by grasping tip cover 270 and rotating tip cover 270. Illustratively, a rotation of tip cover 270 may be configured to rotate fixation mechanism 230 within instrument tip housing 465 until fixation mechanism 230 is temporarily fixed in instrument tip housing 465. In one or more embodiments, a user may remove tip cover 270 from a portion of a first assembled instrument tip 300 by actuating tip cover 270 away from transitory element proximal end 102 after fixation mechanism 230 is temporarily fixed in instrument tip housing 465. Illustratively, the user may perform a first surgical procedure with the first assembled instrument tip 300. In one or more embodiments, the user may remove the first assembled instrument tip 300 from assembled first instrument handle 500 by removing fixation mechanism proximal end 232 from instrument tip housing 465, e.g., the user may remove the first assembled instrument tip 300 from assembled first instrument handle 500 by grasping transitory element 100 and rotating transitory element 100. Illustratively, the user may install a second assembled instrument tip 300 in assembled first instrument handle 500 by inserting fixation mechanism proximal end 232 into instrument tip housing 465. In one or more embodiments, instrument tip housing 465 may be configured to temporarily fix the first assembled instrument tip 300 in instrument tip housing 465, e.g., instrument tip housing 465 may be configured to temporarily fix the first assembled instrument tip 300 in instrument tip housing 465 while the user performs a second surgical procedure.

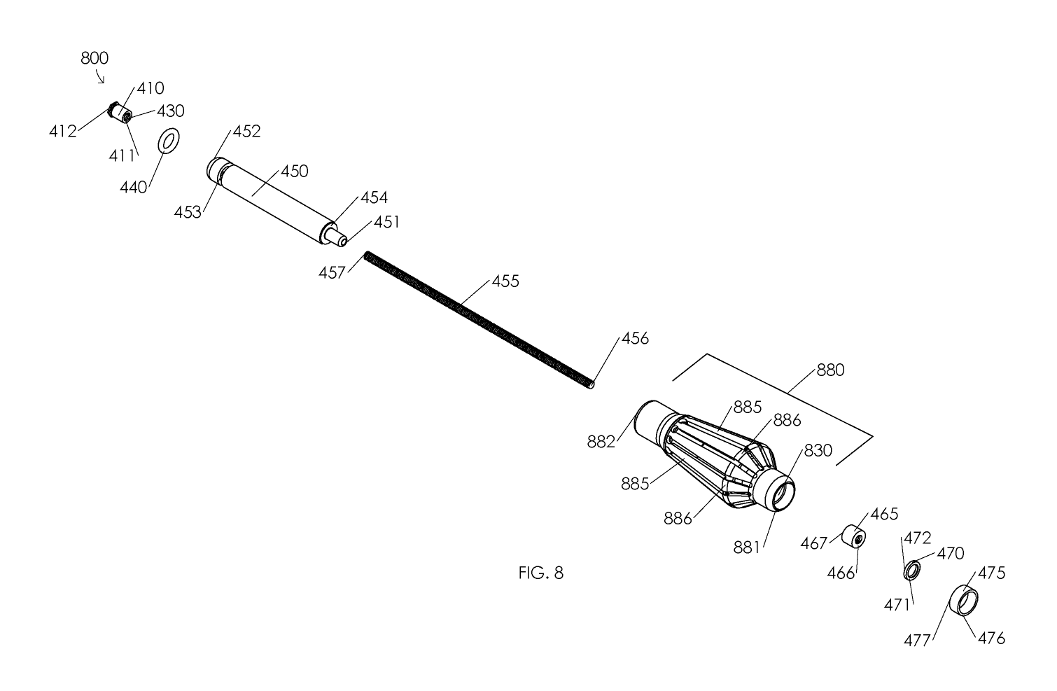

FIG. 8 is a schematic diagram illustrating an exploded view of a second instrument handle assembly 800. Illustratively, a second instrument handle assembly 800 may comprise an end hub 410, a handle identification ring 440, a handle base 450, a threaded rod 455, a second actuation handle 880, an instrument tip housing 465, an instrument tip support ring 470, and a facilitating ring 475. Illustratively, second actuation handle 880 may comprise a second actuation handle distal end 881 and a second actuation handle proximal end 882. In one or more embodiments, second actuation handle 880 may comprise one or more actuation limbs 885. Illustratively, second actuation handle 880 may comprise a plurality of actuation limbs 885. In one or more embodiments, second actuation handle 880 may comprise one or more extension mechanisms 886. Illustratively, second actuation handle 880 may comprise a plurality of extension mechanisms 886. In one or more embodiments, second actuation handle 880 may comprise at least one extension mechanism 886 for each actuation limb 885. Illustratively, second actuation handle 880 may comprise a facilitating ring housing 830, e.g., second actuation handle 880 may comprise a facilitating ring housing 830 disposed between one or more actuation limbs 885. In one or more embodiments, a compression of one or more actuation limbs 885 may be configured to extend second actuation handle distal end 881 relative to second actuation handle proximal end 882. Illustratively, one or more extension mechanisms 886 may be configured to extend second actuation handle distal end 881 relative to second actuation handle proximal end 882. In one or more embodiments, a decompression of one or more actuation limbs 885 may be configured to retract second actuation handle distal end 881 relative to second actuation handle proximal end 882. Illustratively, one or more extension mechanisms 886 may be configured to retract second actuation handle distal end 881 relative to second actuation handle proximal end 882.

In one or more embodiments, second actuation handle 880 may be custom manufactured, e.g., second actuation handle 880 may be manufactured with properties derived from data obtained from a particular surgeon. Illustratively, second actuation handle 880 may be manufactured having an overall outer diameter derived from a particular surgeon's hand dimensions, e.g., a first particular surgeon may have a first particular hand size and a second particular surgeon may have a second particular hand size wherein the second particular hand size is greater than the first particular hand size. In one or more embodiments, a second actuation handle 880 manufactured for the first particular surgeon may have a first overall outer diameter and a second actuation handle 880 manufactured for the second particular surgeon may have a second overall outer diameter wherein the second overall outer diameter is greater than the first overall outer diameter. Illustratively, second actuation handle 880 may be manufactured having an overall length derived from a particular surgeon's hand dimensions, e.g., a first particular surgeon may have a first particular hand size and a second particular surgeon may have a second particular hand size wherein the second particular hand size is greater than the first particular hand size. In one or more embodiments, a second actuation handle 880 manufactured for the first particular surgeon may have a first overall length and a second actuation handle 880 manufactured for the second particular surgeon may have a second overall length wherein the second overall length is greater than the first overall length. Illustratively, second actuation handle 880 may be manufactured to have a particular force applied to one or more actuation limbs 885 to fully extend second actuation handle distal end 881 relative to second actuation handle proximal end 882, e.g., a first surgeon may prefer to apply a first amount of force to one or more actuation limbs 885 to fully extend second actuation handle distal end 881 relative to second actuation handle proximal end 882 and a second surgeon may prefer to apply a second amount of force to one or more actuation limbs 885 to fully extend second actuation handle distal end 881 relative to second actuation handle proximal end 882 wherein the first amount of force is greater than the second amount of force. In one or more embodiments, a second actuation handle 880 manufactured for the first surgeon may have one or more extension mechanisms 886 of a first thickness and a second actuation handle 880 manufactured for the second surgeon may have one or more extension mechanisms 886 of a second thickness wherein the first thickness is greater than the second thickness. Illustratively, second actuation handle 880 may be manufactured from any suitable material, e.g., polymers, metals, metal alloys, etc., or from any combination of suitable materials. In one or more embodiments, second actuation handle 880 may be manufactured by a 3D printing process. For example, second actuation handle 880 may be manufactured by selective laser sintering, selective heat sintering, selective laser melting, electron-beam melting, direct metal laser sintering, electron beam freeform fabrication, etc. Illustratively, second actuation handle 880 may be manufactured by injection molding. In one or more embodiments, second actuation handle 880 may be manufactured by an additive manufacturing process. Illustratively, second actuation handle 880 may be manufacture from a material configured for sterilization in a medical autoclave.

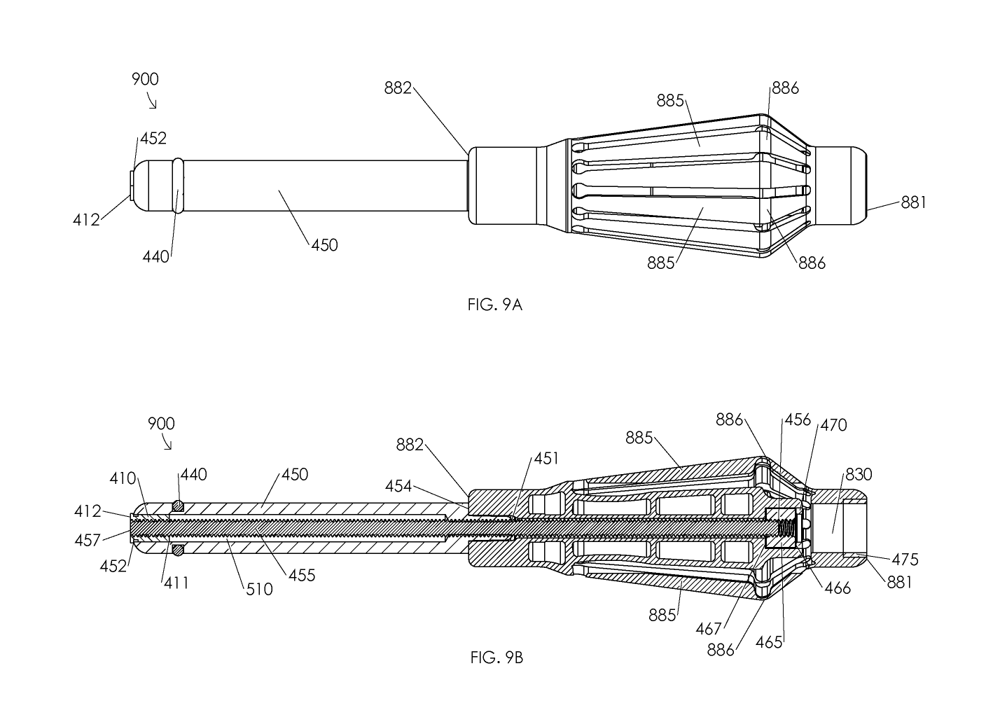

FIGS. 9A and 9B are schematic diagrams illustrating an assembled second instrument handle 900. FIG. 9A illustrates a side view of an assembled second instrument handle 900. FIG. 9B illustrates a cross-sectional in a sagittal plane of an assembled second instrument handle 900. In one or more embodiments, a portion of threaded rod 455 may be disposed in end hub 410, e.g., threaded rod proximal end 457 may be disposed in threaded rod proximal housing 430. Illustratively, a portion of threaded rod 455 may be disposed in end hub 410 wherein threaded rod proximal end 457 may be adjacent to end hub proximal end 412, e.g., a portion of threaded rod 455 may be disposed in end hub 410 wherein threaded rod proximal end 457 may abut end hub proximal end 412. In one or more embodiments, a portion of threaded rod 455 may be fixed in end hub 410, e.g., a portion of threaded rod 455 may be fixed in end hub 410 by an interference fit, an adhesive, an epoxy, a magnetic field, a weld, a threading, etc. Illustratively, a portion of end hub 410 may be disposed in a portion of handle base 450, e.g., end hub distal end 411 may be disposed in handle base inner lumen 510. In one or more embodiments, a portion of end hub 410 may be disposed in a portion of handle base 450 wherein end hub proximal end 412 extends out from handle base proximal end 452. Illustratively, a portion of end hub 410 may be fixed in a portion of handle base 450, e.g., a portion of end hub 410 may be fixed in a portion of handle base 450 by an interference fit, an adhesive, an epoxy, a magnetic field, a weld, a threading, etc.

In one or more embodiments, a portion of threaded rod 455 may be disposed in handle base 450, e.g., a portion of threaded rod 455 may be disposed in handle base inner lumen 510. Illustratively, a portion of threaded rod 455 may be disposed in handle base 450 wherein threaded rod proximal end 457 extends out from handle base proximal end 452. In one or more embodiments, a portion of threaded rod 455 may be disposed in handle base 450 wherein threaded rod distal end 456 extends out from handle base distal end 451. Illustratively, a portion of threaded rod 455 may be disposed in handle base 450 to wherein threaded rod proximal end 457 extends out from handle base proximal end 452 and wherein threaded rod distal end 456 extends out from handle base distal end 451. In one or more embodiments, a portion of threaded rod 455 may be fixed in handle base 450, e.g., a portion of threaded rod 455 may be fixed in handle base 450 by an interference fit, an adhesive, an epoxy, a magnetic field, a weld, a threading, etc.

Illustratively, handle identification ring 440 may be disposed in handle identification ring housing 453. In one or more embodiments, handle identification ring 440 may be fixed in handle identification ring housing 453, e.g., handle identification ring 440 may be fixed in handle identification ring housing 453 by an interference fit, an adhesive, an epoxy, a magnetic field, a weld, a threading, etc. Illustratively, handle identification ring 440 may be configured to indicate one or more properties of assembled second instrument handle 900 to a user, e.g., handle identification ring 440 may be configured to visually indicate one or more properties of assembled second instrument handle 900 to a user. In one or more embodiments, handle identification ring 440 may be configured to indicate a type of cannula that is compatible with assembled second instrument handle 900 to a user, e.g., handle identification ring 440 may be configured to visually indicate a type of cannula that is compatible with assembled second instrument handle 900 to a user. Illustratively, handle identification ring 440 may be configured to indicate a size of cannula that is compatible with assembled second instrument handle 900 to a user, e.g., handle identification ring 440 may be configured to visually indicate a size of cannula that is compatible with assembled second instrument handle 900 to a user. In one or more embodiments, handle identification ring 440 may be configured to correspond to identification ring 280, e.g., a user may compare handle identification ring 440 to identification ring 280 to ensure that a particular assembled instrument tip 300 is configured to connect to a particular assembled second instrument handle 900. Illustratively, a portion of threaded rod 455 may be disposed in a portion of instrument tip housing 465, e.g., threaded rod distal end 456 may be disposed in a portion of instrument tip housing 465. In one or more embodiments, a portion of threaded rod 455 may be fixed in a portion of instrument tip housing 465, e.g., a portion of threaded rod 455 may be fixed in a portion of instrument tip housing 465 by an interference fit, an adhesive, an epoxy, a magnetic field, a weld, a threading, etc.

Illustratively, a portion of handle base 450 may be disposed in a portion of second actuation handle 880, e.g., handle base distal end 451 may be disposed in a portion of second actuation handle 880. In one or more embodiments, a portion of handle base 450 may be disposed in a portion of second actuation handle 880 wherein actuation handle interface 454 may be adjacent to second actuation handle proximal end 882, e.g., a portion of handle base 450 may be disposed in a portion of second actuation handle 880 wherein actuation handle interface 454 may abut second actuation handle proximal end 882. Illustratively, a portion of handle base 450 may be fixed in a portion of second actuation handle 880, e.g., a portion of handle base 450 may be fixed in a portion of second actuation handle 880 by an interference fit, an adhesive, an epoxy, a magnetic field, a weld, a threading, etc. In one or more embodiments, a portion of handle base 450 may be fixed in a portion of second actuation handle 880 by a clearance fit, e.g., a portion of handle base 450 may be fixed in a portion of second actuation handle 880 wherein handle base 450 is configured to rotate relative to second actuation handle 880. Illustratively, a portion of handle base 450 may be fixed in a portion of second actuation handle 880 wherein second actuation handle 880 is configured to rotate relative to handle base 450.

In one or more embodiments, a portion of handle base 450 may be fixed in a portion of second actuation handle 880 by an RC1 clearance fit, an RC2 clearance fit, an RC3 clearance fit, an RC4 clearance fit, an RC5 clearance fit, an RC6 clearance fit, an RC7 clearance fit, an RC8 clearance fit, an RC9 clearance fit, etc.

In one or more embodiments, a portion of threaded rod 455 may be disposed in a portion of second actuation handle 880, e.g., threaded rod distal end 456 may be disposed in a portion of second actuation handle 880. Illustratively, a portion of threaded rod 455 may be disposed in a portion of second actuation handle 880 wherein threaded rod distal end 456 may be disposed between second actuation handle distal end 881 and second actuation handle proximal end 882. In one or more embodiments, a portion of threaded rod 455 may be fixed in a portion of second actuation handle 880, e.g., a portion of threaded rod 455 may be fixed in a portion of second actuation handle 880 by an interference fit, an adhesive, an epoxy, a magnetic field, a weld, a threading, etc. Illustratively, a portion of threaded rod 455 may be fixed in a portion of second actuation handle 880 by a clearance fit, e.g., a portion of threaded rod 455 may be fixed in a portion of second actuation handle 880 wherein threaded rod 455 is configured to rotate relative to second actuation handle 880. In one or more embodiments, a portion of threaded rod 455 may be fixed in a portion of second actuation handle 880 wherein second actuation handle 880 is configured to rotate relative to threaded rod 455. Illustratively, a portion of threaded rod 455 may be fixed in a portion of second actuation handle 880 by an RC1 clearance fit, an RC2 clearance fit, an RC3 clearance fit, an RC4 clearance fit, an RC5 clearance fit, an RC6 clearance fit, an RC7 clearance fit, an RC8 clearance fit, an RC9 clearance fit, etc.

In one or more embodiments, instrument tip housing 465 may be disposed in a portion of second actuation handle 880. Illustratively, instrument tip housing 465 may be disposed in a portion of second actuation handle 880 wherein instrument tip housing distal end 466 may be disposed between second actuation handle distal end 881 and second actuation handle proximal end 882. In one or more embodiments, instrument tip housing 465 may be disposed in a portion of second actuation handle 880 wherein instrument tip housing proximal end 467 may be disposed between second actuation handle distal end 881 and second actuation handle proximal end 882. Illustratively, instrument tip housing 465 may be fixed in a portion of second actuation handle 880, e.g., instrument tip housing 465 may be fixed in a portion of second actuation handle 880 by an interference fit, an adhesive, an epoxy, a magnetic field, a weld, a threading, etc. In one or more embodiments, instrument tip housing 465 may be fixed in a portion of second actuation handle 880 by a clearance fit, e.g., instrument tip housing 465 may be fixed in a portion of second actuation handle 880 wherein instrument tip housing 465 is configured to rotate relative to second actuation handle 880. Illustratively, instrument tip housing 465 may be fixed in a portion of second actuation handle 880 wherein second actuation handle 880 is configured to rotate relative to instrument tip housing 465. In one or more embodiments, instrument tip housing 465 may be fixed in a portion of second actuation handle 880 by an RC1 clearance fit, an RC2 clearance fit, an RC3 clearance fit, an RC4 clearance fit, an RC5 clearance fit, an RC6 clearance fit, an RC7 clearance fit, an RC8 clearance fit, an RC9 clearance fit, etc.

In one or more embodiments, instrument tip support ring 470 may be disposed in a portion of second actuation handle 880. Illustratively, instrument tip support ring 470 may be disposed in a portion of second actuation handle 880 wherein instrument tip support ring proximal end 472 may be adjacent to instrument tip housing distal end 456. In one or more embodiments, instrument tip support ring 470 may be disposed in a portion of second actuation handle 880 wherein instrument tip support ring proximal end 472 may abut instrument tip housing distal end 456. Illustratively, instrument tip support ring 470 may be disposed in a portion of second actuation handle 880 wherein instrument tip support ring distal end 471 may be disposed between second actuation handle distal end 881 and second actuation handle proximal end 882. In one or more embodiments, instrument tip support ring 470 may be disposed in a portion of second actuation handle 880 wherein instrument tip support ring proximal end 472 may be disposed between second actuation handle distal end 881 and second actuation handle proximal end 882. Illustratively, instrument tip support ring 470 may be fixed in a portion of second actuation handle 880, e.g., instrument tip support ring 470 may be fixed in a portion of second actuation handle 880 by an interference fit, an adhesive, an epoxy, a magnetic field, a weld, a threading, etc. In one or more embodiments, instrument tip support ring 470 may be fixed in a portion of second actuation handle 880 by a clearance fit, e.g., instrument tip support ring 470 may be fixed in a portion of second actuation handle 880 wherein instrument tip support ring 470 is configured to rotate relative to second actuation handle 880. Illustratively, instrument tip support ring 470 may be fixed in a portion of second actuation handle 880 wherein second actuation handle 880 is configured to rotate relative to instrument tip support ring 470. In one or more embodiments, instrument tip support ring 470 may be fixed in a portion of second actuation handle 880 by an RC1 clearance fit, an RC2 clearance fit, an RC3 clearance fit, an RC4 clearance fit, an RC5 clearance fit, an RC6 clearance fit, an RC7 clearance fit, an RC8 clearance fit, an RC9 clearance fit, etc.

Illustratively, facilitating ring 475 may be disposed in a portion of second actuation handle 880, e.g., facilitating ring 475 may be disposed in facilitating ring housing 830. In one or more embodiments, facilitating ring 475 may be disposed in a portion of second actuation handle 880 wherein facilitating ring distal end 476 may be adjacent to second actuation handle distal end 881, e.g., facilitating ring 475 may be disposed in a portion of second actuation handle 880 wherein facilitating ring distal end 476 may abut second actuation handle distal end 881. Illustratively, facilitating ring 475 may be fixed in a portion of second actuation handle 880, e.g., facilitating ring 475 may be fixed in a portion of second actuation handle 880 by an interference fit, an adhesive, an epoxy, a magnetic field, a weld, a threading, etc. In one or more embodiments, facilitating ring 475 may be fixed in a portion of second actuation handle 880 by a clearance fit, e.g., facilitating ring 475 may be fixed in a portion of second actuation handle 880 wherein facilitating ring 475 is configured to rotate relative to second actuation handle 880. Illustratively, facilitating ring 475 may be fixed in a portion of second actuation handle 880 wherein second actuation handle 880 is configured to rotate relative to facilitating ring 475. In one or more embodiments, facilitating ring 475 may be fixed in a portion of second actuation handle 880 by an RC1 clearance fit, an RC2 clearance fit, an RC3 clearance fit, an RC4 clearance fit, an RC5 clearance fit, an RC6 clearance fit, an RC7 clearance fit, an RC8 clearance fit, an RC9 clearance fit, etc.

Illustratively, a rotation of handle base 450 relative to second actuation handle 880 may be configured to rotate end hub 410 relative to second actuation handle 880. In one or more embodiments, a rotation of end hub 410 relative to second actuation handle 880 may be configured to rotate threaded rod 455 relative to second actuation handle 880. Illustratively, a rotation of handle base 450 relative to second actuation handle 880 may be configured to rotate threaded rod 455 relative to second actuation handle 880. In one or more embodiments, a rotation of threaded rod 455 relative to second actuation handle 880 may be configured to rotate instrument tip housing 465 relative to second actuation handle 880. Illustratively, a rotation of instrument tip housing 465 relative to second actuation handle 880 may be configured to rotate instrument tip support ring 470 relative to second actuation handle 880. In one or more embodiments, a rotation of handle base 450 relative to second actuation handle 880 may be configured to rotate threaded rod 455, end hub 410, instrument tip housing 465, and instrument tip support ring 470 relative to second actuation handle 880.

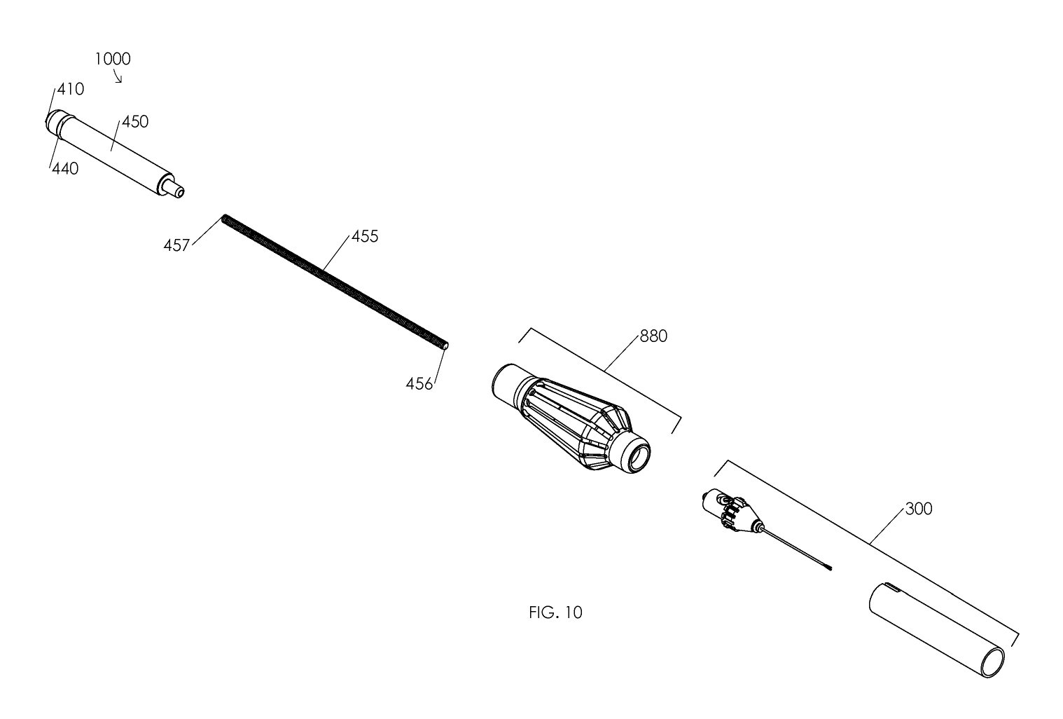

FIG. 10 is a schematic diagram illustrating an exploded view of a second instrument assembly 1000. Illustratively, a second instrument assembly 1000 may comprise an end hub 410, handle identification ring 440, a handle base 450, a threaded rod 455, a second actuation handle 880, and an assembled instrument tip 300.

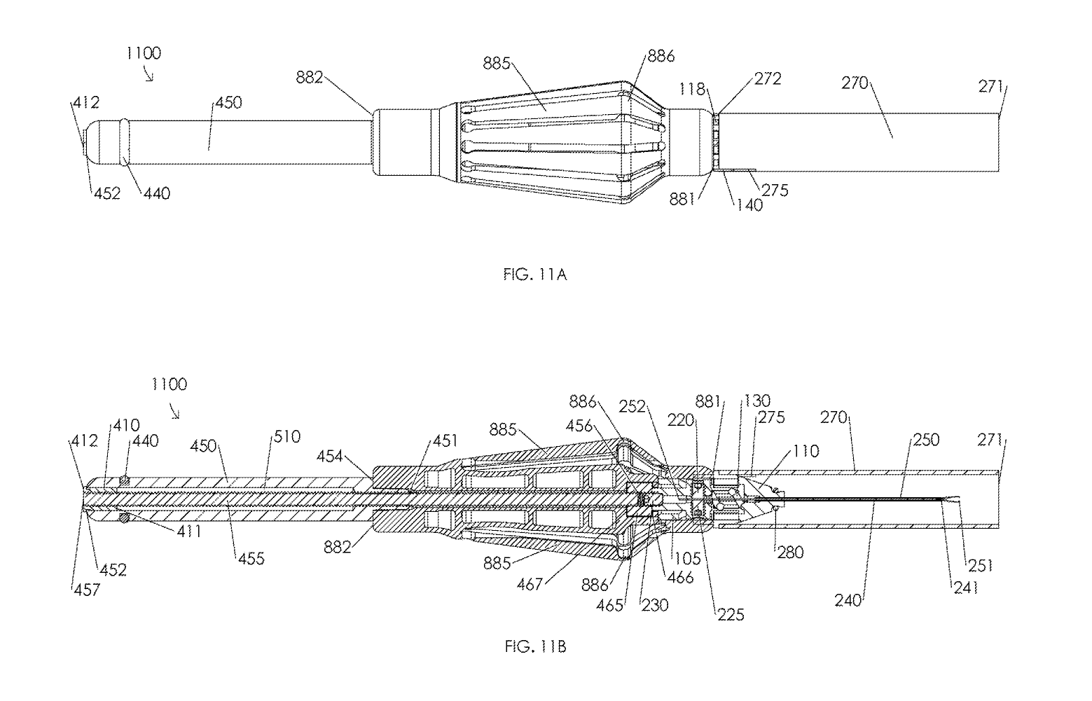

FIGS. 11A and 11B are schematic diagrams illustrating an assembled second instrument 1100. FIG. 11A illustrates a side view of an assembled second instrument 1100. FIG. 11B illustrates a cross-sectional view in a sagittal plane of an assembled second instrument 1100. Illustratively, a portion of fixation mechanism 230 may be disposed in instrument tip housing 465, e.g., fixation mechanism proximal end 232 may be disposed in instrument tip housing 465. In one or more embodiments, a first portion of fixation mechanism 230 may be disposed instrument tip housing 465 wherein a second portion of fixation mechanism 230 may be disposed in fixation mechanism distal receptacle 120, e.g., fixation mechanism proximal end 232 may be disposed in instrument tip housing 465 and fixation mechanism distal end 231 may be disposed in fixation mechanism distal receptacle 120. Illustratively, a first portion of fixation mechanism 230 may be disposed in instrument tip housing 465 wherein a second portion of fixation mechanism 230 may be disposed in fixation mechanism distal housing 121, e.g., fixation mechanism proximal end 232 may be disposed in instrument tip housing 465 and fixation mechanism distal end 321 may be disposed in fixation mechanism distal housing 121. In one or more embodiments, a portion of fixation mechanism 230 may be fixed in instrument tip housing 465, e.g., fixation mechanism proximal end 232 may be fixed in instrument tip housing 465. Illustratively, a portion of fixation mechanism 230 may be fixed in instrument tip housing 465 by an interference fit, an adhesive, an epoxy, a magnetic field, a weld, a threading, etc. In one or more embodiments, a first portion of fixation mechanism 230 may be fixed in instrument tip housing 465 and a second portion of fixation mechanism 230 may be fixed in fixation mechanism distal housing 121, e.g., fixation mechanism proximal end 232 may be fixed in instrument tip housing 465 and fixation mechanism distal end 231 may be fixed in fixation mechanism distal housing 121. In one or more embodiments, instrument tip support ring 470 may be configured to support assembled instrument tip 300, e.g., instrument tip support ring 470 may be configured to prevent assembled instrument tip 300 from vibrating in second actuation handle 880.

Illustratively, fixation mechanism 230 may comprise a setscrew configured to screw into fixation mechanism distal housing 121 and instrument tip housing 465. In one or more embodiments, fixation mechanism 230 may be permanently fixed in fixation mechanism distal housing 121, e.g., fixation mechanism distal end 231 may be fixed in distal housing 121 wherein removing fixation mechanism distal end 231 from distal housing 121 may be configured to damage a portion of transitory element 100. Illustratively, fixation mechanism distal end 231 may be permanently fixed in distal housing 121 wherein removing fixation mechanism distal end 231 from distal housing 121 may be configured to damage fixation mechanism distal receptacle 120. In one or more embodiments, fixation mechanism 230 may be temporarily fixed in instrument tip housing 465, e.g., fixation mechanism proximal end 232 may be fixed in instrument tip housing 465 wherein fixation mechanism proximal end 232 is removable from instrument tip housing 465. Illustratively, a first fixation mechanism proximal end 232 may be temporarily fixed in instrument tip housing 465 wherein the first fixation mechanism proximal end 232 may be removed from instrument tip housing 465 and a second fixation mechanism proximal end 232 may be inserted in instrument tip housing 465. In one or more embodiments, a first fixation mechanism proximal end 232 may be temporarily fixed in instrument tip housing 465 wherein the first fixation mechanism proximal end 232 may be removed from instrument tip housing 465 and a second fixation mechanism proximal end 232 may be temporarily fixed in instrument tip housing 465.

In one or more embodiments, a compression of one or more actuation limbs 885 may be configured to extend second actuation handle distal end 881 relative to second actuation handle proximal end 882. Illustratively, an extension of second actuation handle distal end 881 relative to second actuation handle proximal end 882 may be configured to extend nosecone 110 relative to handle base 105. In one or more embodiments, an extension of nosecone 110 relative to handle base 105 may be configured to extend hypodermic tube 240 relative to blank 250. Illustratively, an extension of hypodermic tube 240 relative to blank 250 may be configured to extend hypodermic tube distal end 241 over a portion of a first instrument jaw 260 and over a portion of a second instrument jaw 260. In one or more embodiments, an extension of hypodermic tube distal end 241 over a portion of a first instrument jaw 260 and over a portion of a second instrument jaw 260 may be configured to reduce a separation distance between the first instrument jaw 260 and the second instrument jaw 260. Illustratively, an extension of nosecone 110 relative to handle base 105 may be configured to expand pressure mechanism 130. In one or more embodiments, pressure mechanism 130 may be configured to provide a force that resists an extension of nosecone 110 relative to handle base 105, e.g., pressure mechanism 130 may be configured to provide a force that facilitates a retraction of nosecone 110 relative to handle base 105.

In one or more embodiments, a decompression of one or more actuation limbs 885 may be configured to retract second actuation handle distal end 881 relative to second actuation handle proximal end 882. Illustratively, a retraction of second actuation handle distal end 881 relative to second actuation handle proximal end 882 may be configured to retract nosecone 110 relative to handle base 105. In one or more embodiments, a retraction of nosecone 110 relative to handle base 105 may be configured to retract hypodermic tube 240 relative to blank 250. Illustratively, a retraction of hypodermic tube 240 relative to blank 250 may be configured to retract hypodermic tube distal end 241 off from a portion of a first instrument jaw 260 and off from a portion of a second instrument jaw 260. In one or more embodiments, a retraction of hypodermic tube distal end 241 off from a portion of a first instrument jaw 260 and off from a portion of a second instrument jaw 260 may be configured to increase a separation distance between the first instrument jaw 260 and the second instrument jaw 260. Illustratively, a retraction of nosecone 110 relative to handle base 105 may be configured to collapse pressure mechanism 130. In one or more embodiments, pressure mechanism 130 may be configured to provide a force that facilitates a retraction of nosecone 110 relative to handle base 105.

Illustratively, a rotation of handle base 450 relative to second actuation handle 880 may be configured to rotate assembled instrument tip 300 relative to second actuation handle 880. In one or more embodiments, a rotation of handle base 450 relative to second actuation handle 880 may be configured to rotate instrument tip housing 465 relative to second actuation handle 880. Illustratively, a rotation of instrument tip housing 465 relative to second actuation handle 880 may be configured to rotate assembled instrument tip 300 relative to second actuation handle 880. In one or more embodiments, second actuation handle 880 may be configured to rotate relative to assembled instrument tip 300. Illustratively, a surgeon may selectively orient first instrument jaw 260 and second instrument jaw 260 in any rotational orientation by rotating handle base 450, e.g., a surgeon may insert first instrument jaw 260 and second instrument jaw 260 through a cannula and into an eye and the surgeon may selectively orient first instrument jaw 260 and second instrument jaw 260 in any rotational orientation within the eye by rotating handle base 450. In one or more embodiments, a surgeon may insert first instrument jaw 260 and second instrument jaw 260 through a cannula and into an eye and the surgeon may selectively orient first instrument jaw 260 and second instrument jaw 260 in any rotational orientation within the eye without rotating second actuation handle 880.

Illustratively, facilitating ring 475 may be configured to facilitate a rotation of assembled instrument tip 300 relative to second actuation handle 880. In one or more embodiments, facilitating ring 475 may be manufactured from a material configured to facilitate a rotation of assembled instrument tip 300 relative to second actuation handle 880. Illustratively, facilitating ring 475 may be manufactured from a material configured to minimize a coefficient of friction between a portion of assembled instrument tip 300 and a portion of facilitating ring 475, e.g., facilitating ring 475 may be manufactured from a material configured to minimize a coefficient of friction between outer base 105 and a portion of facilitating ring 475. In one or more embodiments, facilitating ring 475 may be manufactured from a self-lubricating thermoplastic material. Illustratively, facilitating ring 475 may be manufactured from an oil-impregnated bronze alloy, e.g., facilitating ring 475 may be manufactured from Oilite, SAE 841 bronze, etc. In one or more embodiments, facilitating ring 475 may be manufactured from a material wherein a coefficient of friction between a portion of assembled instrument tip 300 and a portion of facilitating ring 475 is in a range of 0.011 to 0.36, e.g., facilitating ring 475 may be manufactured from a material wherein a coefficient of friction between a portion of assembled instrument tip 300 and a portion of facilitating ring 475 is 0.0311. Illustratively, facilitating ring 475 may be manufactured from a material wherein a coefficient of friction between a portion of assembled instrument tip 300 and a portion of facilitating ring 475 is less than 0.011 or greater than 0.36. In one or more embodiments, facilitating ring 475 may be manufactured from a fluorocarbon material, e.g., facilitating ring 475 may be manufactured from a polytetrafluoroethylene material. Illustratively, facilitating ring 475 may be manufactured from an acetal-based polytetrafluoroethylene material, e.g., facilitating ring 475 may be manufactured from a turcite material. In one or more embodiments, facilitating ring 475 may be manufactured from a material having a density in a range of 0.024 to 0.073 pounds per cubic inch, e.g., facilitating ring 475 may be manufactured from a material having a density of 0.053 pounds per cubic inch. Illustratively, facilitating ring 475 may be manufactured from a material having a density of less than 0.024 pounds per cubic inch or greater than 0.073 pounds per cubic inch. In one or more embodiments, facilitating ring 475 may be manufactured from a material having a hardness in a range of 50 Shore D to 75 Shore D, e.g., facilitating ring 475 may be manufactured from a material having a hardness of 61 Shore D. Illustratively, facilitating ring 475 may be manufactured from a material having a hardness of less than 50 Shore D or greater than 75 Shore D. In one or more embodiments, transitory element 100 may be manufactured from a material having a first hardness and facilitating ring 475 may be manufactured from a material having a second hardness. Illustratively, the first hardness may be greater than the second hardness.

Illustratively, assembled second instrument handle 900 may comprise a reusable instrument and assembled instrument tip 300 may comprise a single-use instrument. In one or more embodiments, a user may install a first assembled instrument tip 300 in assembled second instrument handle 900 by inserting fixation mechanism proximal end 232 into instrument tip housing 465, e.g., a user may install a first assembled instrument tip 300 in assembled second instrument handle 900 by grasping tip cover 270 and rotating tip cover 270. Illustratively, a rotation of tip cover 270 may be configured to rotate fixation mechanism 230 within instrument tip housing 465 until fixation mechanism 230 is temporarily fixed in instrument tip housing 465. In one or more embodiments, a user may remove tip cover 270 from a portion of a first assembled instrument tip 300 by actuating tip cover 270 away from transitory element proximal end 102 after fixation mechanism 230 is temporarily fixed in instrument tip housing 465. Illustratively, the user may perform a first surgical procedure with the first assembled instrument tip 300. In one or more embodiments, the user may remove the first assembled instrument tip 300 from assembled second instrument handle 900 by removing fixation mechanism proximal end 232 from instrument tip housing 465, e.g., the user may remove the first assembled instrument tip 300 from assembled second instrument handle 900 by grasping transitory element 100 and rotating transitory element 100. Illustratively, the user may install a second assembled instrument tip 300 in assembled second instrument handle 900 by inserting fixation mechanism proximal end 232 into instrument tip housing 465. In one or more embodiments, instrument tip housing 465 may be configured to temporarily fix the first assembled instrument tip 300 in instrument tip housing 465, e.g., instrument tip housing 465 may be configured to temporarily fix the first assembled instrument tip 300 in instrument tip housing 465 while the user performs a second surgical procedure.

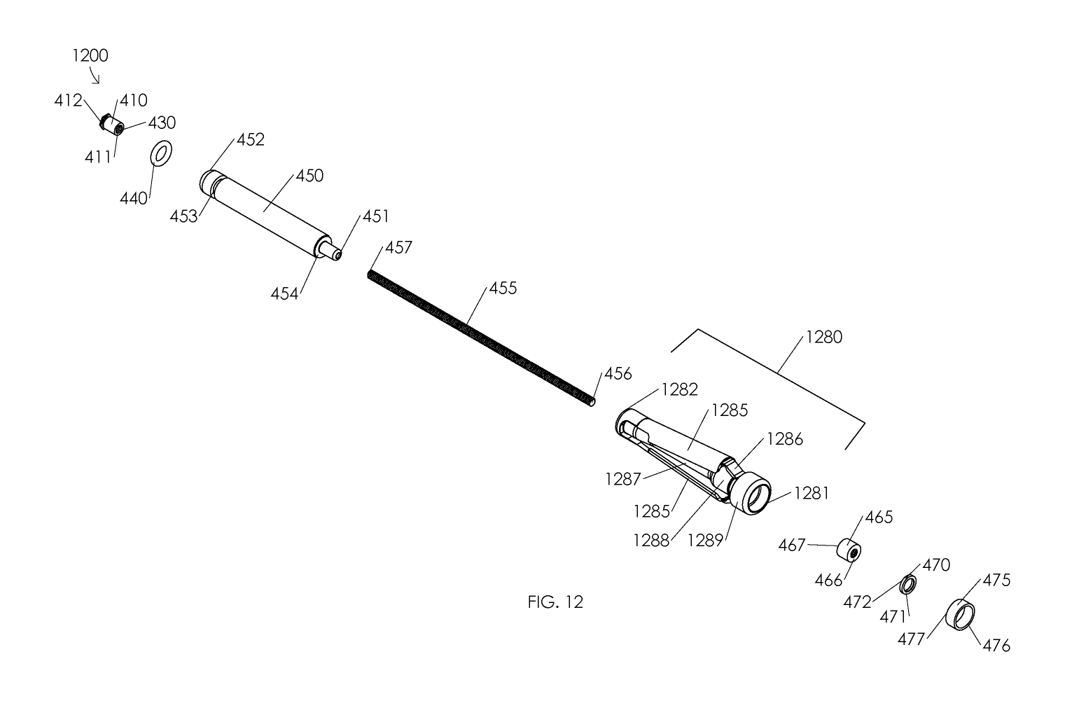

FIG. 12 is a schematic diagram illustrating an exploded view of a third instrument handle assembly 1200. Illustratively, a third instrument handle assembly 1200 may comprise an end hub 410, a handle identification ring 440, a handle base 450, a threaded rod 455, a third actuation handle 1280, an instrument tip housing 465, an instrument tip support ring 470, and a facilitating ring 475. Illustratively, third actuation handle 1280 may comprise a third actuation handle distal end 1281 and a third actuation handle proximal end 1282. In one or more embodiments, third actuation handle 1280 may comprise one or more actuation limbs 1285. Illustratively, third actuation handle 1280 may comprise a plurality of actuation limbs 1285. In one or more embodiments, third actuation handle 1280 may comprise one or more extension mechanisms 1286. Illustratively, third actuation handle 1280 may comprise a plurality of extension mechanisms 1286. In one or more embodiments, third actuation handle 1280 may comprise at least one extension mechanism 1286 for each actuation limb 1285. Illustratively, third actuation handle 1280 may comprise a facilitating ring housing 1289, e.g., third actuation handle 1280 may comprise a facilitating ring housing 1289 disposed between one or more actuation limbs 1285. In one or more embodiments, third actuation handle 1280 may comprise an inner base 1287. Illustratively, third actuation handle 1280 may comprise a housing for instrument tip housing 1288. In one or more embodiments, a compression of one or more actuation limbs 1285 may be configured to extend third actuation handle distal end 1281 relative to third actuation handle proximal end 1282. Illustratively, one or more extension mechanisms 1286 may be configured to extend third actuation handle distal end 1281 relative to third actuation handle proximal end 1282. In one or more embodiments, a decompression of one or more actuation limbs 1285 may be configured to retract third actuation handle distal end 1281 relative to third actuation handle proximal end 1282. Illustratively, one or more extension mechanisms 1286 may be configured to retract third actuation handle distal end 1281 relative to third actuation handle proximal end 1282.

In one or more embodiments, third actuation handle 1280 may be custom manufactured, e.g., third actuation handle 1280 may be manufactured with properties derived from data obtained from a particular surgeon. Illustratively, third actuation handle 1280 may be manufactured having an overall outer diameter derived from a particular surgeon's hand dimensions, e.g., a first particular surgeon may have a first particular hand size and a second particular surgeon may have a second particular hand size wherein the second particular hand size is greater than the first particular hand size. In one or more embodiments, a third actuation handle 1280 manufactured for the first particular surgeon may have a first overall outer diameter and a third actuation handle 1280 manufactured for the second particular surgeon may have a second overall outer diameter wherein the second overall outer diameter is greater than the first overall outer diameter. Illustratively, third actuation handle 1280 may be manufactured having an overall length derived from a particular surgeon's hand dimensions, e.g., a first particular surgeon may have a first particular hand size and a second particular surgeon may have a second particular hand size wherein the second particular hand size is greater than the first particular hand size. In one or more embodiments, a third actuation handle 1280 manufactured for the first particular surgeon may have a first overall length and a third actuation handle 1280 manufactured for the second particular surgeon may have a second overall length wherein the second overall length is greater than the first overall length. Illustratively, third actuation handle 1280 may be manufactured to have a particular force applied to one or more actuation limbs 1285 to fully extend third actuation handle distal end 1281 relative to third actuation handle proximal end 1282, e.g., a first surgeon may prefer to apply a first amount of force to one or more actuation limbs 1285 to fully extend third actuation handle distal end 1281 relative to third actuation handle proximal end 1282 and a second surgeon may prefer to apply a second amount of force to one or more actuation limbs 1285 to fully extend third actuation handle distal end 1281 relative to third actuation handle proximal end 1282 wherein the first amount of force is greater than the second amount of force. In one or more embodiments, a third actuation handle 1280 manufactured for the first surgeon may have one or more extension mechanisms 1286 of a first thickness and a third actuation handle 1280 manufactured for the second surgeon may have one or more extension mechanisms 1286 of a second thickness wherein the first thickness is greater than the second thickness. Illustratively, third actuation handle 1280 may be manufactured from any suitable material, e.g., polymers, metals, metal alloys, etc., or from any combination of suitable materials. In one or more embodiments, third actuation handle 1280 may be manufactured by a 3D printing process. For example, third actuation handle 1280 may be manufactured by selective laser sintering, selective heat sintering, selective laser melting, electron-beam melting, direct metal laser sintering, electron beam freeform fabrication, etc. Illustratively, third actuation handle 1280 may be manufactured by injection molding. In one or more embodiments, third actuation handle 1280 may be manufactured by an additive manufacturing process. Illustratively, third actuation handle 1280 may be manufacture from a material configured for sterilization in a medical autoclave.

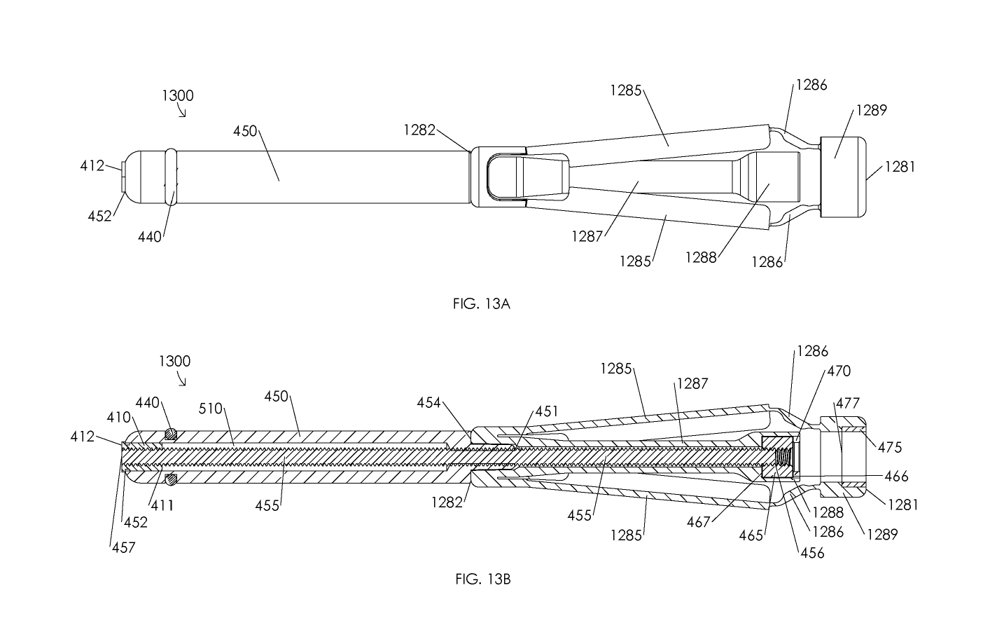

FIGS. 13A and 13B are schematic diagrams illustrating an assembled third instrument handle 1300. FIG. 13A illustrates a side view of an assembled third instrument handle 1300. FIG. 13B illustrates a cross-sectional in a sagittal plane of an assembled third instrument handle 1300. In one or more embodiments, a portion of threaded rod 455 may be disposed in end hub 410, e.g., threaded rod proximal end 457 may be disposed in threaded rod proximal housing 430. Illustratively, a portion of threaded rod 455 may be disposed in end hub 410 wherein threaded rod proximal end 457 may be adjacent to end hub proximal end 412, e.g., a portion of threaded rod 455 may be disposed in end hub 410 wherein threaded rod proximal end 457 may abut end hub proximal end 412. In one or more embodiments, a portion of threaded rod 455 may be fixed in end hub 410, e.g., a portion of threaded rod 455 may be fixed in end hub 410 by an interference fit, an adhesive, an epoxy, a magnetic field, a weld, a threading, etc. Illustratively, a portion of end hub 410 may be disposed in a portion of handle base 450, e.g., end hub distal end 411 may be disposed in handle base inner lumen 510. In one or more embodiments, a portion of end hub 410 may be disposed in a portion of handle base 450 wherein end hub proximal end 412 extends out from handle base proximal end 452. Illustratively, a portion of end hub 410 may be fixed in a portion of handle base 450, e.g., a portion of end hub 410 may be fixed in a portion of handle base 450 by an interference fit, an adhesive, an epoxy, a magnetic field, a weld, a threading, etc.

In one or more embodiments, a portion of threaded rod 455 may be disposed in handle base 450, e.g., a portion of threaded rod 455 may be disposed in handle base inner lumen 510. Illustratively, a portion of threaded rod 455 may be disposed in handle base 450 wherein threaded rod proximal end 457 extends out from handle base proximal end 452. In one or more embodiments, a portion of threaded rod 455 may be disposed in handle base 450 wherein threaded rod distal end 456 extends out from handle base distal end 451. Illustratively, a portion of threaded rod 455 may be disposed in handle base 450 wherein threaded rod proximal end 457 extends out from handle base proximal end 452 and wherein threaded rod distal end 456 extends out from handle base distal end 451. In one or more embodiments, a portion of threaded rod 455 may be fixed in handle base 450, e.g., a portion of threaded rod 455 may be fixed in handle base 450 by an interference fit, an adhesive, an epoxy, a magnetic field, a weld, a threading, etc.

Illustratively, handle identification ring 440 may be disposed in handle identification ring housing 453. In one or more embodiments, handle identification ring 440 may be fixed in handle identification ring housing 453, e.g., handle identification ring 440 may be fixed in handle identification ring housing 453 by an interference fit, an adhesive, an epoxy, a magnetic field, a weld, a threading, etc. Illustratively, handle identification ring 440 may be configured to indicate one or more properties of assembled third instrument handle 1300 to a user, e.g., handle identification ring 440 may be configured to visually indicate one or more properties of assembled third instrument handle 1300 to a user. In one or more embodiments, handle identification ring 440 may be configured to indicate a type of cannula that is compatible with assembled third instrument handle 1300 to a user, e.g., handle identification ring 440 may be configured to visually indicate a type of cannula that is compatible with assembled third instrument handle 1300 to a user. Illustratively, handle identification ring 440 may be configured to indicate a size of cannula that is compatible with assembled third instrument handle 1300 to a user, e.g., handle identification ring 440 may be configured to visually indicate a size of cannula that is compatible with assembled third instrument handle 1300 to a user. In one or more embodiments, handle identification ring 440 may be configured to correspond to identification ring 280, e.g., a user may compare handle identification ring 440 to identification ring 280 to ensure that a particular assembled instrument tip 300 is configured to connect to a particular assembled third instrument handle 1300. Illustratively, a portion of threaded rod 455 may be disposed in a portion of instrument tip housing 465, e.g., threaded rod distal end 456 may be disposed in a portion of instrument tip housing 465. In one or more embodiments, a portion of threaded rod 455 may be fixed in a portion of instrument tip housing 465, e.g., a portion of threaded rod 455 may be fixed in a portion of instrument tip housing 465 by an interference fit, an adhesive, an epoxy, a magnetic field, a weld, a threading, etc.

Illustratively, a portion of handle base 450 may be disposed in a portion of third actuation handle 1280, e.g., handle base distal end 451 may be disposed in a portion of third actuation handle 1280. In one or more embodiments, a portion of handle base 450 may be disposed in a portion of third actuation handle 1280 wherein actuation handle interface 454 may be adjacent to third actuation handle proximal end 1282, e.g., a portion of handle base 450 may be disposed in a portion of third actuation handle 1280 wherein actuation handle interface 454 may abut third actuation handle proximal end 1282. Illustratively, a portion of handle base 450 may be fixed in a portion of third actuation handle 1280, e.g., a portion of handle base 450 may be fixed in a portion of third actuation handle 1280 by an interference fit, an adhesive, an epoxy, a magnetic field, a weld, a threading, etc. In one or more embodiments, a portion of handle base 450 may be fixed in a portion of third actuation handle 1280 by a clearance fit, e.g., a portion of handle base 450 may be fixed in a portion of third actuation handle 1280 wherein handle base 450 is configured to rotate relative to third actuation handle 1280. Illustratively, a portion of handle base 450 may be fixed in a portion of third actuation handle 1280 wherein third actuation handle 1280 is configured to rotate relative to handle base 450. In one or more embodiments, a portion of handle base 450 may be fixed in a portion of third actuation handle 1280 by an RC1 clearance fit, an RC2 clearance fit, an RC3 clearance fit, an RC4 clearance fit, an RC5 clearance fit, an RC6 clearance fit, an RC7 clearance fit, an RC8 clearance fit, an RC9 clearance fit, etc.

In one or more embodiments, a portion of threaded rod 455 may be disposed in a portion of third actuation handle 1280, e.g., threaded rod distal end 456 may be disposed in a portion of third actuation handle 1280. Illustratively, a portion of threaded rod 455 may be disposed in a portion of third actuation handle 1280 wherein threaded rod distal end 456 may be disposed between third actuation handle distal end 1281 and third actuation handle proximal end 1282. In one or more embodiments, a portion of threaded rod 455 may be fixed in a portion of third actuation handle 1280, e.g., a portion of threaded rod 455 may be fixed in a portion of third actuation handle 1280 by an interference fit, an adhesive, an epoxy, a magnetic field, a weld, a threading, etc. Illustratively, a portion of threaded rod 455 may be fixed in a portion of third actuation handle 1280 by a clearance fit, e.g., a portion of threaded rod 455 may be fixed in a portion of third actuation handle 1280 wherein threaded rod 455 is configured to rotate relative to third actuation handle 1280. In one or more embodiments, a portion of threaded rod 455 may be fixed in a portion of third actuation handle 1280 wherein third actuation handle 1280 is configured to rotate relative to threaded rod 455. Illustratively, a portion of threaded rod 455 may be fixed in a portion of third actuation handle 1280 by an RC1 clearance fit, an RC2 clearance fit, an RC3 clearance fit, an RC4 clearance fit, an RC5 clearance fit, an RC6 clearance fit, an RC7 clearance fit, an RC8 clearance fit, an RC9 clearance fit, etc.

In one or more embodiments, instrument tip housing 465 may be disposed in a portion of third actuation handle 1280. Illustratively, instrument tip housing 465 may be disposed in a portion of third actuation handle 1280 wherein instrument tip housing distal end 466 may be disposed between third actuation handle distal end 1281 and third actuation handle proximal end 1282. In one or more embodiments, instrument tip housing 465 may be disposed in a portion of third actuation handle 1280 wherein instrument tip housing proximal end 467 may be disposed between third actuation handle distal end 1281 and third actuation handle proximal end 1282. Illustratively, instrument tip housing 465 may be fixed in a portion of third actuation handle 1280, e.g., instrument tip housing 465 may be fixed in a portion of third actuation handle 1280 by an interference fit, an adhesive, an epoxy, a magnetic field, a weld, a threading, etc. In one or more embodiments, instrument tip housing 465 may be fixed in a portion of third actuation handle 1280 by a clearance fit, e.g., instrument tip housing 465 may be fixed in a portion of third actuation handle 1280 wherein instrument tip housing 465 is configured to rotate relative to third actuation handle 1280. Illustratively, instrument tip housing 465 may be fixed in a portion of third actuation handle 1280 wherein third actuation handle 1280 is configured to rotate relative to instrument tip housing 465. In one or more embodiments, instrument tip housing 465 may be fixed in a portion of third actuation handle 1280 by an RC1 clearance fit, an RC2 clearance fit, an RC3 clearance fit, an RC4 clearance fit, an RC5 clearance fit, an RC6 clearance fit, an RC7 clearance fit, an RC8 clearance fit, an RC9 clearance fit, etc.

In one or more embodiments, instrument tip support ring 470 may be disposed in a portion of third actuation handle 1280, e.g., instrument tip support ring 470 may be disposed in housing for instrument tip housing 1288. Illustratively, instrument tip support ring 470 may be disposed in a portion of third actuation handle 1280 wherein instrument tip support ring proximal end 472 may be adjacent to instrument tip housing distal end 456. In one or more embodiments, instrument tip support ring 470 may be disposed in a portion of third actuation handle 1280 wherein instrument tip support ring proximal end 472 may abut instrument tip housing distal end 456. Illustratively, instrument tip support ring 470 may be disposed in a portion of third actuation handle 1280 wherein instrument tip support ring distal end 471 may be disposed between third actuation handle distal end 1281 and third actuation handle proximal end 1282. In one or more embodiments, instrument tip support ring 470 may be disposed in a portion of third actuation handle 1280 wherein instrument tip support ring proximal end 472 may be disposed between third actuation handle distal end 1281 and third actuation handle proximal end 1282. Illustratively, instrument tip support ring 470 may be fixed in a portion of third actuation handle 1280, e.g., instrument tip support ring 470 may be fixed in a portion of third actuation handle 1280 by an interference fit, an adhesive, an epoxy, a magnetic field, a weld, a threading, etc. In one or more embodiments, instrument tip support ring 470 may be fixed in a portion of third actuation handle 1280 by a clearance fit, e.g., instrument tip support ring 470 may be fixed in a portion of third actuation handle 1280 wherein instrument tip support ring 470 is configured to rotate relative to third actuation handle 1280. Illustratively, instrument tip support ring 470 may be fixed in a portion of third actuation handle 1280 wherein third actuation handle 1280 is configured to rotate relative to instrument tip support ring 470. In one or more embodiments, instrument tip support ring 470 may be fixed in a portion of third actuation handle 1280 by an RC1 clearance fit, an RC2 clearance fit, an RC3 clearance fit, an RC4 clearance fit, an RC5 clearance fit, an RC6 clearance fit, an RC7 clearance fit, an RC8 clearance fit, an RC9 clearance fit, etc.

Illustratively, facilitating ring 475 may be disposed in a portion of third actuation handle 1280, e.g., facilitating ring 475 may be disposed in facilitating ring housing 1289. In one or more embodiments, facilitating ring 475 may be disposed in a portion of third actuation handle 1280 wherein facilitating ring distal end 476 may be adjacent to third actuation handle distal end 1281, e.g., facilitating ring 475 may be disposed in a portion of third actuation handle 1280 wherein facilitating ring distal end 476 may abut third actuation handle distal end 1281. Illustratively, facilitating ring 475 may be fixed in a portion of third actuation handle 1280, e.g., facilitating ring 475 may be fixed in a portion of third actuation handle 1280 by an interference fit, an adhesive, an epoxy, a magnetic field, a weld, a threading, etc. In one or more embodiments, facilitating ring 475 may be fixed in a portion of third actuation handle 1280 by a clearance fit, e.g., facilitating ring 475 may be fixed in a portion of third actuation handle 1280 wherein facilitating ring 475 is configured to rotate relative to third actuation handle 1280. Illustratively, facilitating ring 475 may be fixed in a portion of third actuation handle 1280 wherein third actuation handle 1280 is configured to rotate relative to facilitating ring 475. In one or more embodiments, facilitating ring 475 may be fixed in a portion of third actuation handle 1280 by an RC1 clearance fit, an RC2 clearance fit, an RC3 clearance fit, an RC4 clearance fit, an RC5 clearance fit, an RC6 clearance fit, an RC7 clearance fit, an RC8 clearance fit, an RC9 clearance fit, etc.

Illustratively, a rotation of handle base 450 relative to third actuation handle 1280 may be configured to rotate end hub 410 relative to third actuation handle 1280. In one or more embodiments, a rotation of end hub 410 relative to third actuation handle 1280 may be configured to rotate threaded rod 455 relative to third actuation handle 1280. Illustratively, a rotation of handle base 450 relative to third actuation handle 1280 may be configured to rotate threaded rod 455 relative to third actuation handle 1280. In one or more embodiments, a rotation of threaded rod 455 relative to third actuation handle 1280 may be configured to rotate instrument tip housing 465 relative to third actuation handle 1280. Illustratively, a rotation of instrument tip housing 465 relative to third actuation handle 1280 may be configured to rotate instrument tip support ring 470 relative to third actuation handle 1280. In one or more embodiments, a rotation of handle base 450 relative to third actuation handle 1280 may be configured to rotate threaded rod 455, end hub 410, instrument tip housing 465, and instrument tip support ring 470 relative to third actuation handle 1280.

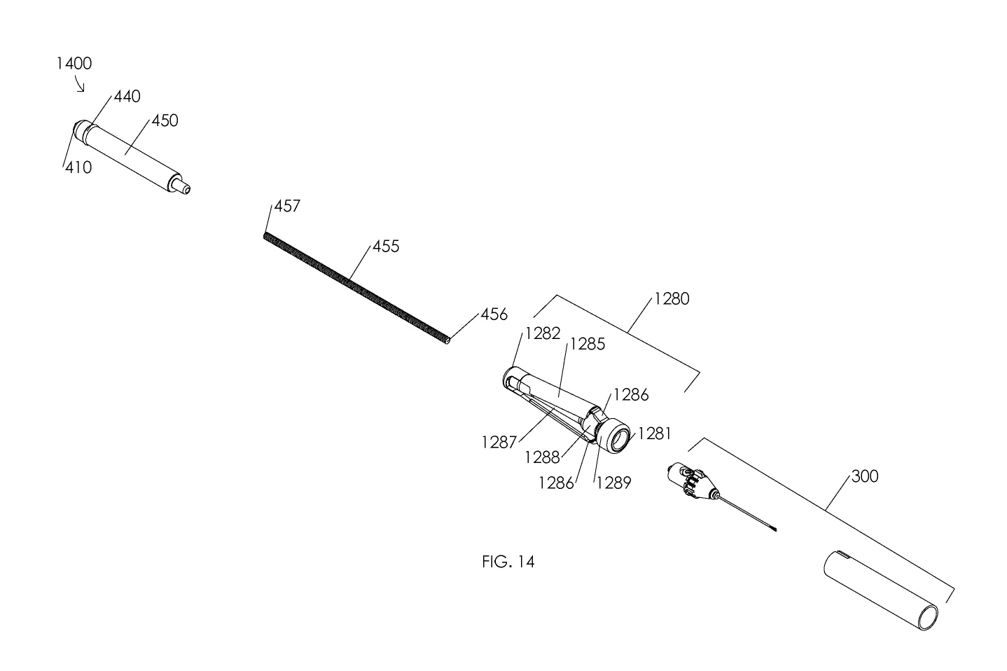

FIG. 14 is a schematic diagram illustrating an exploded view of a third instrument assembly 1400. Illustratively, a third instrument assembly 1400 may comprise an end hub 410, handle identification ring 440, a handle base 450, a threaded rod 455, a third actuation handle 1280, and an assembled instrument tip 300.

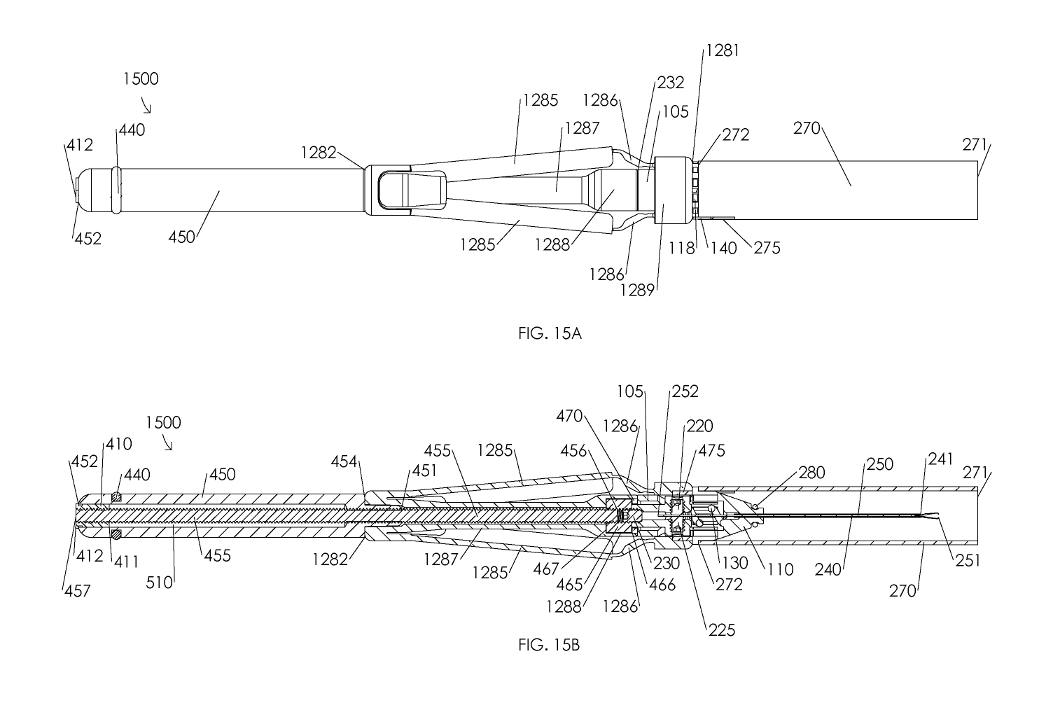

FIGS. 15A and 15B are schematic diagrams illustrating an assembled third instrument 1500. FIG. 15A illustrates a side view of an assembled third instrument 1500. FIG. 15B illustrates a cross-sectional view in a sagittal plane of an assembled third instrument 1500. Illustratively, a portion of fixation mechanism 230 may be disposed in instrument tip housing 465, e.g., fixation mechanism proximal end 232 may be disposed in instrument tip housing 465. In one or more embodiments, a first portion of fixation mechanism 230 may be disposed instrument tip housing 465 wherein a second portion of fixation mechanism 230 may be disposed in fixation mechanism distal receptacle 120, e.g., fixation mechanism proximal end 232 may be disposed in instrument tip housing 465 and fixation mechanism distal end 231 may be disposed in fixation mechanism distal receptacle 120. Illustratively, a first portion of fixation mechanism 230 may be disposed in instrument tip housing 465 wherein a second portion of fixation mechanism 230 may be disposed in fixation mechanism distal housing 121, e.g., fixation mechanism proximal end 232 may be disposed in instrument tip housing 465 and fixation mechanism distal end 321 may be disposed in fixation mechanism distal housing 121. In one or more embodiments, a portion of fixation mechanism 230 may be fixed in instrument tip housing 465, e.g., fixation mechanism proximal end 232 may be fixed in instrument tip housing 465. Illustratively, a portion of fixation mechanism 230 may be fixed in instrument tip housing 465 by an interference fit, an adhesive, an epoxy, a magnetic field, a weld, a threading, etc. In one or more embodiments, a first portion of fixation mechanism 230 may be fixed in instrument tip housing 465 and a second portion of fixation mechanism 230 may be fixed in fixation mechanism distal housing 121, e.g., fixation mechanism proximal end 232 may be fixed in instrument tip housing 465 and fixation mechanism distal end 231 may be fixed in fixation mechanism distal housing 121. In one or more embodiments, instrument tip support ring 470 may be configured to support assembled instrument tip 300, e.g., instrument tip support ring 470 may be configured to prevent assembled instrument tip 300 from vibrating in third actuation handle 1280.

Illustratively, fixation mechanism 230 may comprise a setscrew configured to screw into fixation mechanism distal housing 121 and instrument tip housing 465. In one or more embodiments, fixation mechanism 230 may be permanently fixed in fixation mechanism distal housing 121, e.g., fixation mechanism distal end 231 may be fixed in distal housing 121 wherein removing fixation mechanism distal end 231 from distal housing 121 may be configured to damage a portion of transitory element 100. Illustratively, fixation mechanism distal end 231 may be permanently fixed in distal housing 121 wherein removing fixation mechanism distal end 231 from distal housing 121 may be configured to damage fixation mechanism distal receptacle 120. In one or more embodiments, fixation mechanism 230 may be temporarily fixed in instrument tip housing 465, e.g., fixation mechanism proximal end 232 may be fixed in instrument tip housing 465 wherein fixation mechanism proximal end 232 is removable from instrument tip housing 465. Illustratively, a first fixation mechanism proximal end 232 may be temporarily fixed in instrument tip housing 465 wherein the first fixation mechanism proximal end 232 may be removed from instrument tip housing 465 and a second fixation mechanism proximal end 232 may be inserted in instrument tip housing 465. In one or more embodiments, a first fixation mechanism proximal end 232 may be temporarily fixed in instrument tip housing 465 wherein the first fixation mechanism proximal end 232 may be removed from instrument tip housing 465 and a second fixation mechanism proximal end 232 may be temporarily fixed in instrument tip housing 465.

In one or more embodiments, a compression of one or more actuation limbs 1285 may be configured to extend third actuation handle distal end 1281 relative to third actuation handle proximal end 1282. Illustratively, an extension of third actuation handle distal end 1281 relative to third actuation handle proximal end 1282 may be configured to extend nosecone 110 relative to handle base 105. In one or more embodiments, an extension of nosecone 110 relative to handle base 105 may be configured to extend hypodermic tube 240 relative to blank 250. Illustratively, an extension of hypodermic tube 240 relative to blank 250 may be configured to extend hypodermic tube distal end 241 over a portion of a first instrument jaw 260 and over a portion of a second instrument jaw 260. In one or more embodiments, an extension of hypodermic tube distal end 241 over a portion of a first instrument jaw 260 and over a portion of a second instrument jaw 260 may be configured to reduce a separation distance between the first instrument jaw 260 and the second instrument jaw 260. Illustratively, an extension of nosecone 110 relative to handle base 105 may be configured to expand pressure mechanism 130. In one or more embodiments, pressure mechanism 130 may be configured to provide a force that resists an extension of nosecone 110 relative to handle base 105, e.g., pressure mechanism 130 may be configured to provide a force that facilitates a retraction of nosecone 110 relative to handle base 105.

In one or more embodiments, a decompression of one or more actuation limbs 1285 may be configured to retract third actuation handle distal end 1281 relative to third actuation handle proximal end 1282. Illustratively, a retraction of third actuation handle distal end 1281 relative to third actuation handle proximal end 1282 may be configured to retract nosecone 110 relative to handle base 105. In one or more embodiments, a retraction of nosecone 110 relative to handle base 105 may be configured to retract hypodermic tube 240 relative to blank 250. Illustratively, a retraction of hypodermic tube 240 relative to blank 250 may be configured to retract hypodermic tube distal end 241 off from a portion of a first instrument jaw 260 and off from a portion of a second instrument jaw 260. In one or more embodiments, a retraction of hypodermic tube distal end 241 off from a portion of a first instrument jaw 260 and off from a portion of a second instrument jaw 260 may be configured to increase a separation distance between the first instrument jaw 260 and the second instrument jaw 260. Illustratively, a retraction of nosecone 110 relative to handle base 105 may be configured to collapse pressure mechanism 130. In one or more embodiments, pressure mechanism 130 may be configured to provide a force that facilitates a retraction of nosecone 110 relative to handle base 105.

Illustratively, a rotation of handle base 450 relative to third actuation handle 1280 may be configured to rotate assembled instrument tip 300 relative to third actuation handle 1280. In one or more embodiments, a rotation of handle base 450 relative to third actuation handle 1280 may be configured to rotate instrument tip housing 465 relative to third actuation handle 1280. Illustratively, a rotation of instrument tip housing 465 relative to third actuation handle 1280 may be configured to rotate assembled instrument tip 300 relative to third actuation handle 1280. In one or more embodiments, third actuation handle 1280 may be configured to rotate relative to assembled instrument tip 300. Illustratively, a surgeon may selectively orient first instrument jaw 260 and second instrument jaw 260 in any rotational orientation by rotating handle base 450, e.g., a surgeon may insert first instrument jaw 260 and second instrument jaw 260 through a cannula and into an eye and the surgeon may selectively orient first instrument jaw 260 and second instrument jaw 260 in any rotational orientation within the eye by rotating handle base 450. In one or more embodiments, a surgeon may insert first instrument jaw 260 and second instrument jaw 260 through a cannula and into an eye and the surgeon may selectively orient first instrument jaw 260 and second instrument jaw 260 in any rotational orientation within the eye without rotating third actuation handle 1280.

Illustratively, facilitating ring 475 may be configured to facilitate a rotation of assembled instrument tip 300 relative to third actuation handle 1280. In one or more embodiments, facilitating ring 475 may be manufactured from a material configured to facilitate a rotation of assembled instrument tip 300 relative to third actuation handle 1280. Illustratively, facilitating ring 475 may be manufactured from a material configured to minimize a coefficient of friction between a portion of assembled instrument tip 300 and a portion of facilitating ring 475, e.g., facilitating ring 475 may be manufactured from a material configured to minimize a coefficient of friction between outer base 105 and a portion of facilitating ring 475. In one or more embodiments, facilitating ring 475 may be manufactured from a self-lubricating thermoplastic material. Illustratively, facilitating ring 475 may be manufactured from an oil-impregnated bronze alloy, e.g., facilitating ring 475 may be manufactured from Oilite, SAE 841 bronze, etc. In one or more embodiments, facilitating ring 475 may be manufactured from a material wherein a coefficient of friction between a portion of assembled instrument tip 300 and a portion of facilitating ring 475 is in a range of 0.011 to 0.36, e.g., facilitating ring 475 may be manufactured from a material wherein a coefficient of friction between a portion of assembled instrument tip 300 and a portion of facilitating ring 475 is 0.0311. Illustratively, facilitating ring 475 may be manufactured from a material wherein a coefficient of friction between a portion of assembled instrument tip 300 and a portion of facilitating ring 475 is less than 0.011 or greater than 0.36. In one or more embodiments, facilitating ring 475 may be manufactured from a fluorocarbon material, e.g., facilitating ring 475 may be manufactured from a polytetrafluoroethylene material. Illustratively, facilitating ring 475 may be manufactured from an acetal-based polytetrafluoroethylene material, e.g., facilitating ring 475 may be manufactured from a turcite material. In one or more embodiments, facilitating ring 475 may be manufactured from a material having a density in a range of 0.024 to 0.073 pounds per cubic inch, e.g., facilitating ring 475 may be manufactured from a material having a density of 0.053 pounds per cubic inch. Illustratively, facilitating ring 475 may be manufactured from a material having a density of less than 0.024 pounds per cubic inch or greater than 0.073 pounds per cubic inch. In one or more embodiments, facilitating ring 475 may be manufactured from a material having a hardness in a range of 50 Shore D to 75 Shore D, e.g., facilitating ring 475 may be manufactured from a material having a hardness of 61 Shore D. Illustratively, facilitating ring 475 may be manufactured from a material having a hardness of less than 50 Shore D or greater than 75 Shore D. In one or more embodiments, transitory element 100 may be manufactured from a material having a first hardness and facilitating ring 475 may be manufactured from a material having a second hardness. Illustratively, the first hardness may be greater than the second hardness.

Illustratively, assembled third instrument handle 1300 may comprise a reusable instrument and assembled instrument tip 300 may comprise a single-use instrument. In one or more embodiments, a user may install a first assembled instrument tip 300 in assembled third instrument handle 1300 by inserting fixation mechanism proximal end 232 into instrument tip housing 465, e.g., a user may install a first assembled instrument tip 300 in assembled third instrument handle 1300 by grasping tip cover 270 and rotating tip cover 270. Illustratively, a rotation of tip cover 270 may be configured to rotate fixation mechanism 230 within instrument tip housing 465 until fixation mechanism 230 is temporarily fixed in instrument tip housing 465. In one or more embodiments, a user may remove tip cover 270 from a portion of a first assembled instrument tip 300 by actuating tip cover 270 away from transitory element proximal end 102 after fixation mechanism 230 is temporarily fixed in instrument tip housing 465. Illustratively, the user may perform a first surgical procedure with the first assembled instrument tip 300. In one or more embodiments, the user may remove the first assembled instrument tip 300 from assembled third instrument handle 1300 by removing fixation mechanism proximal end 232 from instrument tip housing 465, e.g., the user may remove the first assembled instrument tip 300 from assembled third instrument handle 1300 by grasping transitory element 100 and rotating transitory element 100. Illustratively, the user may install a second assembled instrument tip 300 in assembled third instrument handle 1300 by inserting fixation mechanism proximal end 232 into instrument tip housing 465. In one or more embodiments, instrument tip housing 465 may be configured to temporarily fix the first assembled instrument tip 300 in instrument tip housing 465, e.g., instrument tip housing 465 may be configured to temporarily fix the first assembled instrument tip 300 in instrument tip housing 465 while the user performs a second surgical procedure.

The foregoing description has been directed to particular embodiments of this invention. It will be apparent; however, that other variations and modifications may be made to the described embodiments, with the attainment of some or all of their advantages. Specifically, it should be noted that the principles of the present invention may be implemented in any system. Furthermore, while this description has been written in terms of a surgical instrument, the teachings of the present invention are equally suitable to any systems where the functionality may be employed. Therefore, it is the object of the appended claims to cover all such variations and modifications as come within the true spirit and scope of the invention.

What is claimed is:

1. An instrument comprising:
a transitory element having a transitory element distal end and a transitory element proximal end;
a hypodermic tube having a hypodermic tube distal end and a hypodermic tube proximal end wherein the hypodermic tube proximal end is disposed in the transitory element and wherein the hypodermic tube distal end extends out from the transitory element distal end;
a blank having a blank distal end and a blank proximal end, the blank disposed in the transitory element and the hypodermic tube wherein the blank distal end extends out from the hypodermic tube distal end;
an actuation handle having an actuation handle distal end and an actuation handle proximal end;
at least one actuation limb of the actuation handle;
at least one extension mechanism of the actuation handle;
an instrument tip housing having an instrument tip housing distal end and an instrument tip housing proximal end wherein the instrument tip housing is disposed in the actuation handle;
a handle base having a handle base distal end and a handle base proximal end;
a rod having a rod distal end and a rod proximal end, the rod disposed in the handle base, the actuation handle, and the instrument tip housing; and
a fixation mechanism having a fixation mechanism distal end and a fixation mechanism proximal end, the fixation mechanism disposed in the instrument tip housing and the transitory element.

2. The instrument of claim 1 wherein the transitory element is configured to rotate relative to the actuation handle.

3. The instrument of claim 1 wherein the actuation handle is configured to rotate relative to the transitory element.

4. The instrument of claim 1 wherein a rotation of the handle base relative to the actuation handle is configured to rotate the transitory element relative to the actuation handle.

5. The instrument of claim 1 further comprising:
a facilitating ring having a facilitating ring distal end and a facilitating ring proximal end wherein the facilitating ring is disposed in the actuation handle.

6. The instrument of claim 1 further comprising:
an instrument tip support ring having an instrument tip support ring distal end and an instrument tip support ring proximal end wherein the instrument tip support ring is disposed in the actuation handle.

7. The instrument of claim 1 further comprising:
an end hub having an end hub distal end and an end hub proximal end wherein the end hub is disposed in the handle base.

8. The instrument of claim 1 further comprising:
a tip cover having a tip cover distal end and a tip cover distal end wherein the tip cover is disposed over the blank and the hypodermic tube.

9. The instrument of claim 1 further comprising:
a pressure mechanism of the transitory element.

10. The instrument of claim 1 further comprising:
an instrument jaw of the blank.

11. The instrument of claim 10 wherein the instrument jaw is a forceps jaw.

12. The instrument of claim 10 wherein the instrument jaw is a scissors blade.

13. The instrument of claim 1 further comprising:
a superior projection of the transitory element.

14. The instrument of claim 1 further comprising:
an inferior projection of the transitory element.

15. The instrument of claim 1 further comprising:
an inner bore of the transitory element; and
an inner bore distal taper of the transitory element.

16. The instrument of claim 1 wherein at least a portion of the rod is threaded.

17. The instrument of claim 1 wherein a compression of at least one actuation limb is configured to extend the actuation handle distal end relative to the actuation handle proximal end.

18. The instrument of claim 1 wherein a compression of at least one actuation limb is configured to extend the hypodermic tube relative to the blank.

19. The instrument of claim 1 wherein a decompression of at least one actuation limb is configured to retract the actuation handle distal end relative to the actuation handle proximal end.

20. The instrument of claim 1 wherein a decompression of at least one actuation limb is configured to retract the hypodermic tube relative to the blank.
